(19) United States
(12) Patent Application Publication
(10) Pub. No.: US 2017/0335383 A1
(43) Pub. Date: Nov. 23, 2017

(54) LINEAR-EXPO-LINEAR PCR (LEL-PCR)

(71) Applicant: Bdrandeis University, Waltham, MA (US)

(72) Inventors: Lawrence J. Wangh, Auburndale, MA (US); J. A. Sanchez, Framingham, MA (US)

(21) Appl. No.: 15/328,798

(22) PCT Filed: Jul. 24, 2015
(86) PCT No.: PCT/US2015/041943

§ 371 Date: Jan. 24, 2017

Related U.S. Application Data
(60) Provisional application No. 62/028,511, filed on Jul. 24, 2014.

(51) Int. Cl. C12Q 1/68; C12P 19/34

(52) U.S. Cl. CPC C12Q 1/6858; C12P 19/34

(57) ABSTRACT

Disclosed herein is a nucleic acid amplification process referred to as Linear-Expo-Linear Polymerase Chain Reaction (LEL-PCR).

### RELATED APPLICATIONS

This application claims the benefit of priority to U.S. Patent Application Ser. No. 62/028,511, filed Jul. 24, 2014, the contents of which is hereby incorporated by reference.

### BACKGROUND

The polymerase chain reaction (PCR) is a biochemical technique developed in the 1980s that allows for the amplification of a nucleic acid sequence, generating thousands to millions of copies referred to as amplification products or amplicons. Since its initial conception, PCR technology has dramatically advanced through the advent of many complementary technologies, including the use of heat-stable DNA polymerases, the invention of the thermocycler, and the development of numerous detection technologies, including technologies that allow for the detection of amplicon formation in real-time without opening the tube in which the amplification reaction is taking place.

Since its discovery, PCR amplification and associated technologies have been applied to a wide range of fields, from basic research, to molecular diagnostics and infectious agent detection. Indeed, nucleic acid amplification technologies have become a critical tool in the health care system. However, current nucleic acid amplification technologies can be prone to errors. Because the target nucleic acid sequence is amplified at an exponential rate, amplification errors are rapidly propagated and can dramatically skew results. Thus, there is a great need for improved methods for accurate and reliable nucleic acid amplification.

### SUMMARY

In certain aspects, disclosed herein is a novel amplification process referred to as Linear-Expo-Linear Polymerase Chain Reaction (“LEL-PCR”). In LEL-PCR, a target nucleic acid sequence in a target nucleic acid molecule is initially subjected to a linear amplification reaction to generate an initial amplification product. Following linear amplification, the initial amplification product is subjected to a LATE-PCR amplification reaction in which it is first exponentially amplified and then linearly amplified, thereby producing a final amplification product that can be subsequently or simultaneously detected. In some embodiments, the method is performed using Temperature Imprecise PCR (TI-PCR).

In some embodiments, provided herein is a method of amplifying a target nucleic acid sequence in a target nucleic acid molecule comprising the steps of: (a) forming a reaction solution comprising the target nucleic acid molecule, a forward primer, a reverse primer and amplification reagents; (b) subjecting the reaction solution to conditions such that a linear amplification reaction is performed on the target nucleic acid molecule producing a first single-stranded amplification product comprising the forward primer and a sequence complementary to the target nucleic acid sequence; (c) subjecting the reaction solution to conditions such that an exponential amplification reaction is performed on the first single-stranded amplification product producing a double-stranded nucleic acid amplification product comprising a first strand comprising the forward primer, a sequence complementary to the target nucleic acid sequence and a sequence complementary to the reverse primer, and comprising a second strand comprising the reverse primer, the target nucleic acid sequence and a sequence complementary to the forward primer; and (d) subjecting the reaction solution to conditions such that a linear amplification reaction is performed on the first strand of the double-stranded amplification product producing a second single-stranded amplification product comprising the reverse primer, the target nucleic acid sequence and a sequence complementary to the forward primer.

In some embodiments, the method includes the step of forming a reaction solution. In some embodiments, the reaction solution includes a target nucleic acid molecule, a forward primer, a reverse primer and amplification reagents. In some embodiments, the forward primer has partial complementarity to a nucleic acid sequence on the 3′ end of the target nucleic acid sequence. In some embodiments, the reverse primer has partial identity to a nucleic acid sequence on the 5′ end of the target nucleic acid sequence. In some embodiments, the melting temperature for the reverse primer on the target nucleic acid sequence is lower than the melting temperature for the forward primer on the target nucleic acid sequence (e.g., at least 5° C. or 10° C. lower than the melting temperature for the forward primer on the target nucleic acid sequence). In some embodiments, the reverse primer is present in the reaction solution at a higher concentration than the forward primer (e.g., at least 2-fold higher or at least 5-fold higher). In some embodiments, the forward primer is a SuperSelective primer. In some embodiments, the reverse primer comprises a 3′ region that is identical to the 5′ end of the target nucleic acid sequence and a 5′ region that is different from the 5′ end of the target nucleic acid sequence. In some embodiments, the reaction solution includes a reagent for detecting the formation of an amplification product (e.g., a detectably labeled probe, such as a molecular beacon). In some embodiments, the detection reagent comprises a Lights-On probe and a Lights-Off probe or a Lights-Off Only probe and a dsDNA fluorescent dye. In some embodiments, the reaction solution includes a Temperature Dependent Reagent. In some embodiments, the reaction solution includes a limiting primer blocking oligonucleotide.

In some embodiments, the method includes the step of subjecting the reaction solution to one or more linear amplification cycle (e.g., 1-10 amplification cycles). In some embodiments, the linear amplification cycles comprise an annealing temperature that is lower than the melting temperature of the forward primer on the target nucleic acid sequence and higher than the melting temperature of the reverse primer on the target nucleic acid sequence.

In some embodiments, the method includes subjecting the reaction solution to one or more low annealing temperature amplification cycles (e.g., a single low annealing temperature cycle). In some embodiments, the low annealing temperature amplification cycles comprise an annealing temperature that is lower than the melting temperature of the reverse primer on the target nucleic acid sequence.

In some embodiments, the method includes subjecting the reaction solution to one or more LATE-PCR amplification cycles (e.g., at least 30 cycles, such as 60 cycles). In some embodiments, the LATE-PCR amplification cycles comprise an annealing temperature that is above the melting temperatures for the forward primer and the reverse primer on the target nucleic acid sequence and below the melting temperature for the forward primer and the reverse primer on perfectly complementary nucleic acid sequences.

In some embodiments, the method includes a step of detecting the amplification product formed in the LATE-PCR amplification cycles. In some embodiments, the amplification product is detected in real-time. In some embodiments, the amplification product is detected following completion of the LEL-PCR amplification process. In some embodiments, the amplification product is detected without opening the reaction tube containing the reaction solution.

In certain aspects, provided herein is a kit for performing a Linear-Expo-Linear (LEL-PCR) amplification on a target nucleic acid sequence. In some embodiments, the kit includes a forward primer, a reverse primer and instructions for performing a LEL-PCR amplification. In some embodiments, the forward primer has partial complementarity to nucleic acid sequence on the 3′ end of the target nucleic acid sequence. In some embodiments, the reverse primer has partial identity to a nucleic acid sequence on the 5′ end of the target nucleic acid sequence. In some embodiments, the melting temperature for the reverse primer on the target nucleic acid sequence is lower than the melting temperature for the forward primer on the target nucleic acid sequence. In some embodiments, the kit further comprises amplification reagents. In some embodiments, the kit further comprises a reagent for detecting a single-stranded amplification product. In some embodiments, the kit further comprises a Temperature Dependent Reagent. In some embodiments, the kit further comprises a limiting primer blocking oligonucleotide.

### BRIEF DESCRIPTION OF FIGURES

FIG. 1 is a schematic depicting the use of selective limiting primers with LATE-PCR and Lights-On/Lights-Off probes.

FIG. 2 is a schematic depicting the use of a SuperSelective limiting primer with LATE-PCR and Lights-On/Lights-Off probes.

FIG. 3 is a schematic depicting the use of DISSECT with LATE-PCR and Lights-On/Lights-Off probes.

FIG. 4 shows preferential amplification of 10,000 copies of mutant EGFR L858R DNA (Curve 1) relative to 10,000 copies of wild-type EGFR DNA (Curve 2).

FIG. 5 shows preferential amplification of 10,000 copies of mutant BRAF V600E DNA (Curve 3) relative to 10,000 copies of wild-type BRAF DNA (Curve 4).

FIG. 6 shows that increasing concentrations of EP003 did not appreciably affect amplification of EGFR L858R mutant targets (Curve 5, all EP003 concentrations) but preferentially delayed the amplification of wild-type EGFR targets (Curve 6, 0 nM EP003; Curve 7, 25 nM EP003; Curve 8, 50 nM EP003; Curve 9, 100 nM EP003).

FIG. 7 shows that increasing concentrations of EP003 did not appreciably affect amplification of BRAF V600E mutant targets (Curve 10, all EP003 concentrations) but preferentially delayed the amplification of wild-type BRAF targets (Curve 11, 0 nM EP003; Curve 12, 25 nM EP003; Curve 13, 50 nM EP003; Curve 14, 100 nM EP003).

FIG. 8 shows an exemplary temperature cycling profile used for SuperSelective primers in certain amplification reactions.

FIG. 9 is a schematic depicting the use of SuperSelective primers according to certain embodiments of the methods disclosed herein. When a SuperSelective primer is hybridized to an original target molecule, the bridge sequence in the primer remains single stranded and forms a bubble. Once a SuperSelective primers is successfully extended on the matched target sequence the resulting amplification product incorporates the entire SuperSelective primer sequence (including the bridge sequence). As a result, the melting temperature of the SuperSelective primer on the amplification product target is higher than its melting temperature on the original target sequence.

FIG. 10 is a schematic depicting the application of SuperSelective primers to LATE-PCR according to certain embodiments of the methods disclosed herein. SuperSelective primer and the reverse primers are converted to LATE-PCR primers and a 5′ tail non-complementary to the original target sequence is added to the LATE-PCR reverse primer such that, once incorporated into an amplification product, the melting temperature of the fully complementary reverse primer on the amplification product target is higher on than the melting temperature on the original target.

FIG. 11 shows an exemplary temperature cycling profile for SuperSelective primers in certain LATE-PCR amplification reactions.

FIG. 12 shows an exemplary temperature profile for SuperSelective primers in certain LEL-PCR amplification reactions. In this embodiment, a limiting SuperSelective primer undergoes several cycles of linear amplification with an annealing temperature of below the melting temperature of the SuperSelective primer on the original target sequence and above the melting temperature of the reverse primer on the original target sequence (e.g., 71° C.). A single cycle is then performed with an annealing temperature of below the melting temperature for the reverse primer on the original target sequence (e.g., 60° C.). This is followed by multiple cycles with an annealing temperature of above the melting temperatures of the SuperSelective primer and the reverse primer on the original target sequence, but below the melting temperatures of the SuperSelective primer and the reverse primer on the amplification product sequence (e.g., 75° C.) to enable amplification of amplification product targets first exponentially and then, once the limiting SuperSelective primer has been exhausted, linearly.

FIG. 13 shows preferential amplification of three replicates of 10,000 copies of matched targets (Curves 15) relative to only one out of three replicates of 10,000 copies of mismatched targets (Curves 16) after a single round of linear extension of the limiting SuperSelective primer at 70° C.

FIG. 14 shows that increasing the number of linear amplification cycles for the LATE-PCR SuperSelective limiting primer from one to ten allows better amplification of the matched targets (Curves 17) but enough mismatched targets hybridize under these conditions to allow amplification of all three replicates (Curves 18).

FIG. 15 shows that addition of 25 nM of the Reagent 2 increased the selectivity of the LATE-PCR SuperSelective primers by 0.8 Ct values. The delta Ct value between the matched target (Curves 19) and the mismatched target (Curves 20) was 7.3 cycles compared to the delta Ct value between the matched target+25 nM Reagent 2 (Curves 21) and the mismatched target+25 nM Reagent 2 (Curves 22).

FIG. 16 shows that Reagent 2 present in the samples from FIG. 16 can be readily visualized by virtue of its own fluorescence (Cal Orange, in this particular example). Curves 23 correspond to reactions without Reagent 2; Curves 24 corresponds to reactions with 25 nM Reagent 2.

FIG. 17 shows that the probe readily distinguished selectively amplified amplicons containing a simulated internal unmethylated site from those containing the same simulated site but methylated. Curves 25 correspond to amplicons with an internal unmethylated site, Curves 26 correspond to amplicons with an internal methylated site.

FIG. 18 shows that Hairpin Reagent 1 (Curve 27) prevented amplification over a range of temperature. Control reactions with Taq DNA polymerase+Taq DNA polymerase antibody demonstrate that failure to amplify was due to Hairpin Reagent 1 controlling the activity of Taq DNA polymerase at the annealing temperature (Curve 28).

FIG. 19 is a schematic depicting Temperature Imprecise PCR (TI-PCR) according to certain embodiments of the methods described herein.

FIG. 20 is an alignment of the rpoB gene showing exemplary primer positions.

FIG. 21 is an alignment of the rpoB gene showing exemplary primer positions.

FIG. 22 shows the results of two monoplex LEL-PCR amplification reactions in which both the LEL-PCR limiting primer and the LEL-PCR excess primer have 5′ tail sequences that are not complementary to the target at the start of amplification, but become complementary to the amplified product as a result of amplification. Curve 29 is the LEL-PCR 1 SYBR Green amplification. Curve 30 is the LEL-PCR 2 SYBR Green amplification; curve 31 is the LEL-PCR 1 probe hybridization signal, Cal Red 610 channel. Curve 32 is the LEL-PCR 2 probe hybridization signal, Cal Orange 560 channel. Each curve corresponds to the average of three replicates samples.

FIG. 23 shows that 100 nM ThermaGo-3 effectively suppresses primer dimer formation in no target control LEL-PCR amplifications. Curve 33 is the SYBR Green amplification in the absence of ThermaGo-3. Curve 34 is the SYBR Green amplification in the presence of 100 nM ThermaGo-3. Each curve corresponds to the average of three replicates samples.

FIG. 24 shows that LEL-PCR can be performed in multiplex reactions and that addition of 100 nM ThermaGo-3 improves amplification in LEL-PCR multiplex reactions. Curve 35 is the LEL-PCR 1 probe hybridization signal without ThermaGo-3, Cal Red 610 channel. Curve 36 is the LEL-PCR 1 probe hybridization signal in the presence of 100 nM ThermaGo-3, Cal Red 610 channel. Curve 37 is the LEL-PCR 2 probe hybridization signal without ThermaGo-3, Cal Orange 560 channel. Curve 38 is the LEL-PCR 2 probe hybridization signal in the presence of 100 nM ThermaGo-3, Cal Orange 560 channel. Each curve corresponds to the average of three replicates samples.

FIG. 25 shows the use of complementary oligonucleotides that bind to the 3′ end of the LEL-PCR limiting primer to prevent mis-priming during a LEL-PCR amplification. Curve 39 is the LEL-PCR 1 probe hybridization signal in the absence of limiting primer blocking oligonucleotides, Cal Red 610 channel. Curve 40 is the LEL-PCR 1 probe hybridization signal in the presence of 100 nM limiting primer blocking oligonucleotides, Cal Red 610 channel. Each curve corresponds to the average of three replicates samples.

FIG. 26 shows that LEL-PCR can successfully be performed on GC-rich genomic templates despite the challenges associated with amplification from such targets. Curve 41 is the SYBR green amplification curve produced during the LEL-PCR amplification of a GC-rich template. Curve 42 is probe hybridization signal produced during the LEL-PCR amplification of a GC-rich template, Quasar 670 channel. Each curve corresponds to the average of three replicates samples.

### DETAILED DESCRIPTION

### General

Provided herein is a novel amplification process referred to as Linear-Expo-Linear Polymerase Chain Reaction (“LEL-PCR”). In LEL-PCR, a target nucleic acid sequence in a target nucleic acid molecule is initially subjected to a linear amplification reaction to generate an initial amplification product. Following linear amplification, the initial amplification product is subjected to a LATE-PCR amplification reaction in which it is first exponentially amplified and then linearly amplified, thereby producing a final amplification product that can be subsequently or simultaneously detected. In some embodiments, the method is performed using Temperature Imprecise PCR (TI-PCR). Also provided herein are kits for performing LEL-PCR.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the terms “hybridize” or “hybridization” refer to the hydrogen bonding of complementary DNA and/or RNA sequences to form a duplex molecule. As used herein, hybridization generally takes place under conditions that can be adjusted to a level of stringency that reduces or even prevents base-pairing between a first oligonucleotide primer or oligonucleotide probe and a target sequence, if the complementary sequences are mismatched by as little as one base-pair. In a closed tube reaction, the level of stringency can be adjusted by changing temperature and, as a result, the hybridization of a primer or a probe to a target can occur or not occur depending on temperature. Thus, for example, a probe or a primer that is mismatched to a target can be caused to hybridize to the target by sufficiently lowering the temperature of the solution.

As used herein, the Tm or melting temperature of two oligonucleotides is the temperature at which 50% of the oligonucleotide/targets are bound and 50% of the oligonucleotide target molecules are not bound. Tm values are concentration dependent and are affected by the concentration of monovalent, divalent cations in a reaction mixture. Tm can be determined empirically or calculated using the nearest neighbor formula, as described in Santa Lucia, J. PNAS (USA) 95:1460-1465 (1998), which is hereby incorporated by reference. LEL-PCR takes into account the concentration-adjusted melting temperature of the limiting primer at the start of amplification, Tm_{[0]}^{L}, the concentration-adjusted melting temperature of the excess primer at the start of amplification, Tm_{[0]}^{X}, and the concentration-adjusted melting temperature of the single-stranded amplification product, Tm_{A}. For LEL-PCR primers, Tm_{[0]} can be determined empirically, as necessary when non-natural nucleotides are used, or calculated according to the nearest neighbor method using a salt concentration adjustment. For LEL-PCR the melting temperature of the amplicon is calculated using the formula: Tm=81.5+0.41 (% G+% C)−500/L+16.6 log [M]/(1+0.7[M]), where L is the length in nucleotides and [M] is the molar concentration of monovalent cations.

As used herein, the term “limiting primer blocking oligonucleotide” refers to an oligonucleotide that hybridizes to the 3′ end of a LEL-PCR limiting primer during a LEL-PCR annealing/extension step to form a blunt-ended double stranded hybrid that inhibits binding of the LEL-PCR limiting primer to nonspecific targets during this step, thereby preventing mis-priming.

As used herein, the term “Linear-After-The Exponential PCR” or “LATE-PCR” refers to a non-symmetric PCR method that utilizes unequal concentrations of primers and yields single-stranded primer-extension products (referred to herein as amplification products or amplicons). LATE-PCR is described, for example, in U.S. Pat. Nos. 7,198,897 and 8,367,325, each of which is incorporated by reference in its entirety.

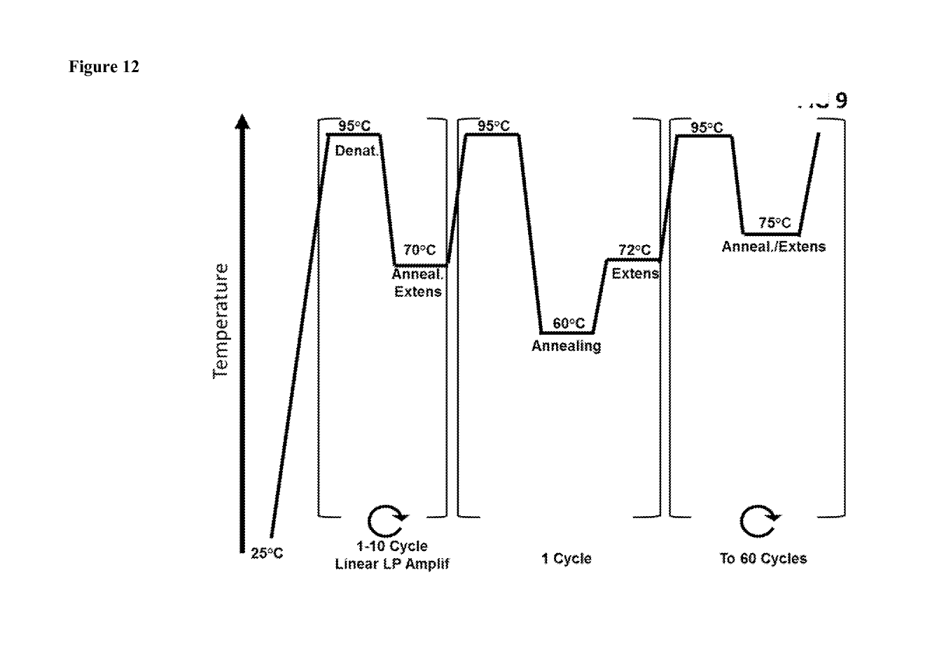

As used herein, the term “Linear-Expo-Linear PCR” or “LEL-PCR” refers to a PCR method in which a target nucleic acid sequence undergoes an initial linear amplification process producing an amplification product that is then selectively subjected to LATE-PCR. An exemplary LEL-PCR process is depicted in FIG. 12.

As used herein, Low-Tm probes and Superlow-Tm probes are fluorescently tagged, electrically tagged or quencher tagged oligonucleotides that have a Tm of at least 5° C. below the mean primer annealing temperature during exponential amplification of a LATE-PCR amplification. In some embodiments sets of signaling and quencher Low-Tm and Superlow-Tm probes are included in LATE-PCR amplification mixtures prior to the start of amplification. There are many possible designs of Low-Tm and Superlow-Tm probes. Molecular beacons, for example, can be designed to be Low-Tm probes by designing them with shorter stems and loops compared standard molecular beacons that hybridize to target strands at or above the primer annealing temperature of the reaction.

As used herein, the term “Lights-On/Lights-Off probes” refers to a probe set that hybridize to adjacent nucleic acid sequences on the single-stranded DNA target to be detected Lights-On/Lights-Off probe technology is more fully described in PCT application No. PCT/US10/53569, hereby incorporated by reference in its entirety.

As used herein, Lights-Off Only probes are probes labeled with a non-fluorescent quencher moiety (e.g., a Black Hole quencher) that hybridize to a single-stranded DNA target to be detected. Lights-Off Only probes are used in combination with a fluorescent dye that binds preferentially to double-stranded DNA (e.g., SYBR® Green dye) to detect single-stranded amplification products (e.g., single-stranded DNA products produced by LATE-PCR). This is done by subjecting an amplified sample containing the fluorescent ds-DNA dye and the Lights-Off Only probe at multiple temperatures that are below the melting temperature of the probe to excitation at a wavelength appropriate for stimulating the dye and detecting emission at a wavelength appropriate for detecting emission from the dsDNA-dye. Lights-Off Only probe technology is more fully described in U.S. Provisional Application No. 61/702,019, hereby incorporated by reference in its entirety.

As used herein, the term “Temperature Dependent Reagent” refers to a modified double-stranded or hairpin oligonucleotide that increases amplification efficiency, decreases mis-priming and/or decreases primer-dimer formation during PCR amplification reactions. Temperature Dependent Reagents are further described in U.S. Patent application publication 2012/0088275 and U.S. Pat. No. 7,517,977, and U.S. Provisional Patent application Nos. 62/094,597 and 62/136,048, each of which is hereby incorporated by reference in its entirety.

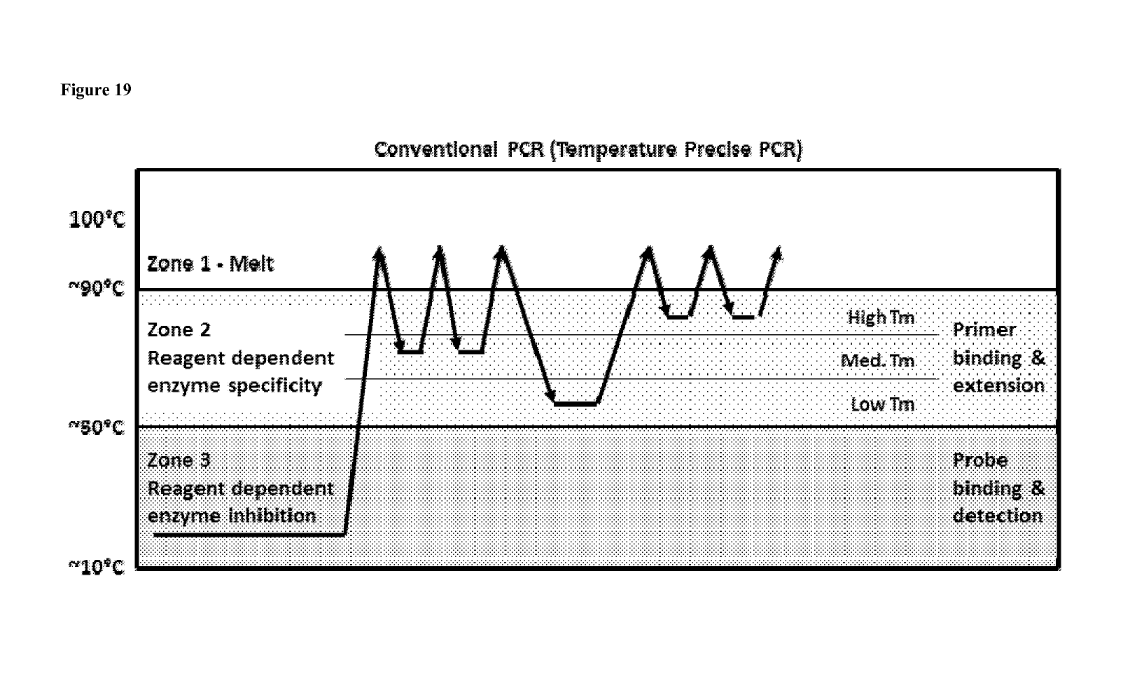

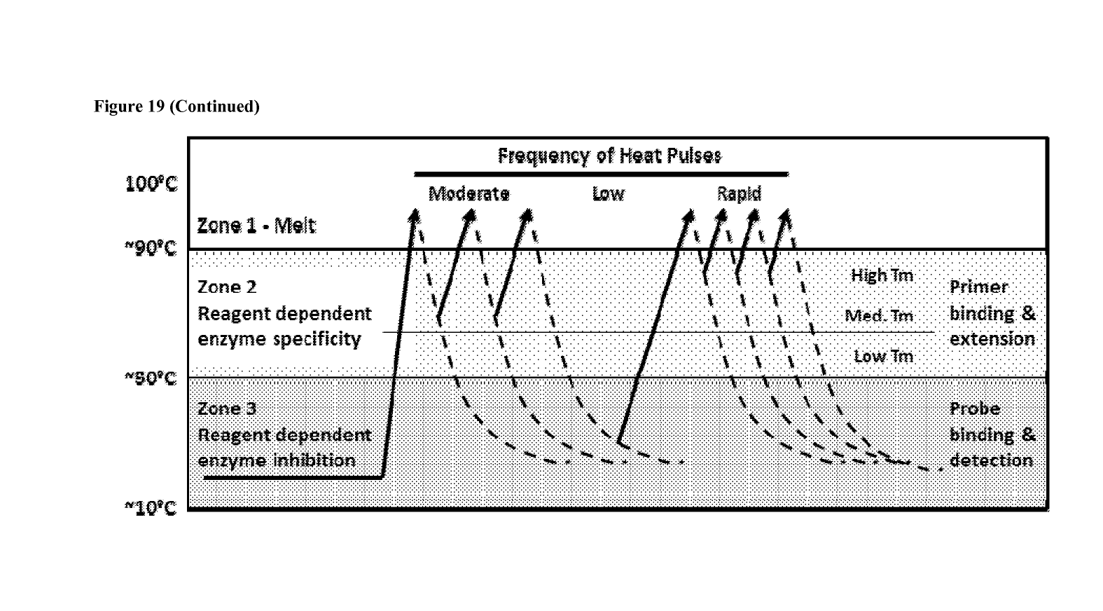

As used herein, the term “Temperature Imprecise PCR” or “TI-PCR” refers to a PCR amplification method in which the temperature of the reaction vessel is elevated by heating at time-adjustable intervals for time-adjustable lengths of time and in which the temperature of the reaction vessel is decreased via passive cooling for time-adjustable intervals. The principles of TI-PCR can be applied to other PCR amplification techniques, including LATE-PCR and LEL-PCR. An exemplary TI-PCR process is depicted in FIG. 19.

### LEL-PCR

In certain embodiments, provided herein are methods of performing LEL-PCR. LEL-PCR is a PCR method in which a sample containing a target nucleic acid is subjected to amplification conditions such that the target nucleic acid sequence first undergoes one or more rounds (e.g., 1-10 rounds) of a linear amplification process to produce a single-stranded amplification product containing a sequence complementary to the target nucleic acid sequence. The sample is then subjected to conditions such that the single-stranded amplification product is subjected to one or more rounds of an exponential amplification process to produce a double-stranded amplification product in which a first strand contains a sequence complementary to the target nucleic acid sequence and a second strand contains a sequence corresponding to the target nucleic acid sequence and complementary to the sequence of the first amplification product strand. Following exponential amplification, the double-stranded amplification product is then subject to a linear amplification process in which a second single-stranded amplification product is generated. In certain embodiments, the second single-stranded amplification product will contain a sequence corresponding to the target sequence.

In certain embodiments, LEL-PCR is performed by combining a first primer, referred to as the forward primer and a second primer, referred to as a reverse primer, with a target nucleic acid and amplification reagents. The first and second primers are designed such that they are not perfectly complementary to the target nucleic acid sequence. For example, in some embodiments, the first and/or second primer have an internal non-complementary region (e.g., SuperSelective primers), have a non-complementary 5′ region, and/or contain sequence mismatches with respect to the target nucleic acid sequence. In some embodiments, the forward primer is a SuperSelective primer and the reverse primer has a non-complementary 5′ region. The first and second primers therefore have a first melting temperature on the imperfectly complementary target sequence and a second, higher melting temperature on a perfectly complementary sequence. In certain embodiments, the melting temperature of the forward primer on the target nucleic acid sequence is higher than the melting temperature of the reverse primer on the target nucleic acid sequence. In some embodiments, the melting temperature of the reverse primer on a perfectly complementary sequence is higher than the melting temperature of the forward primer on the target nucleic acid sequence. In some embodiments, the forward primer is a limiting primer and is included in the reaction solution at a lower molar concentration than the reverse primer. An exemplary LEL-PCR process is depicted in FIG. 12.

In some embodiments, the LEL-PCR process includes an initial linear amplification phase. In certain embodiments, the initial linear amplification phase includes one or more linear amplification cycles. In some embodiments, the initial linear amplification phase includes at least 1, 2, 3, 4, 5, 6, 7, 8, 9 or 10 cycles. In some embodiments, the initial linear amplification phase includes between 1 and 10 cycles. In some embodiments, each cycle of the linear amplification phase includes at least a denaturation step and an annealing step. In some embodiments, each cycle also includes an extension step. In some embodiments, the annealing step and the extension step are combined into a single step. In some embodiments, the denaturation step is performed at a temperature above the melting temperature of the forward primer on the target nucleic acid sequence. In some embodiments, the denaturation step is performed at a temperature above the melting temperature of the forward primer for a perfectly complementary sequence. In some embodiments, the denaturation step is performed at between 90° C. and 100° C. In some embodiments, the denaturation step is performed at a temperature of about 95° C. In some embodiments, the annealing step is performed at a temperature that is below the melting temperature of the forward primer on the target nucleic acid sequence but above the melting temperature of the reverse primer on the target nucleic acid sequence. In some embodiments, the annealing step is combined with an extension step, and the extension step is performed at a temperature at which a polymerase enzyme in the reaction solution is active (e.g., between about 70° C. and 75° C.). In some embodiments, the annealing step is followed by an extension step that is performed at a temperature at which a polymerase enzyme in the reaction solution is active (e.g., between about 70° C. and 75° C.).

In some embodiments, the initial linear amplification phase is followed by one or more low annealing temperature cycles. In some embodiments, the initial linear amplification phase is followed by a single low annealing temperature cycle. In some embodiments, the low annealing temperature cycle includes at least a denaturation step and an annealing step. In some embodiments, the cycle also includes an extension step. In some embodiments, the annealing step and the extension step are combined into a single step. In some embodiments, the denaturation step is performed at a temperature above the melting temperature of the forward primer on the target nucleic acid sequence. In some embodiments, the denaturation step is performed at a temperature above the melting temperature of the forward primer for a perfectly complementary sequence. In some embodiments, the denaturation step is performed at between 90° C. and 100° C. In some embodiments, the denaturation step is performed at a temperature of about 95° C. In some embodiments, the annealing step is performed at a temperature that is below the melting temperature of the reverse primer on the target nucleic acid sequence. In some embodiments, the annealing step is combined with an extension step, and the extension step is performed at a temperature at which a polymerase enzyme in the reaction solution is active (e.g., between about 70° C. and 75° C.). In some embodiments, the annealing step is followed by an extension step that is performed at a temperature at which a polymerase enzyme in the reaction solution is active (e.g., between about 70° C. and 75° C.).

In some embodiments, the low annealing temperature cycle is followed by performance of a LATE-PCR phase that includes an exponential amplification phase and a linear amplification phase. In certain embodiments, the LATE-PCR phase includes one or more amplification cycles. In some embodiments, LATE-PCR phase includes at least 10, 15, 20, 25, 30, 35, 40, 45, 50, 55 or 60 cycles. In some embodiments, the early cycles of the LATE-PCR phase, when both the forward primer and the reverse primer are present, both strands of a target sequence are amplified exponentially, as occurs in conventional PCR, but in the later cycles, when only one primer is present, only one strand of the target sequence amplified linearly. In some embodiments, the forward primer is limiting. In some embodiments, each cycle of the LATE-PCR phase includes at least a denaturation step and an annealing step. In some embodiments, each cycle also includes an extension step. In some embodiments, the annealing step and the extension step are combined into a single step. In some embodiments, the denaturation step is performed at a temperature above the melting temperature of both the forward primer and the reverse primer on perfectly complementary nucleic acid sequences. In some embodiments, the denaturation step is performed at between 90° C. and 100° C. In some embodiments, the denaturation step is performed at a temperature of about 95° C. In some embodiments, the annealing step is performed at a temperature that is above the melting temperature of the forward primer on the target nucleic acid sequence but below the melting temperature of the forward primer on a perfectly complementary nucleic acid sequence. In some embodiments, the annealing step is performed at a temperature that is above the melting temperature of the reverse primer on the target nucleic acid sequence but below the melting temperature of the reverse primer on a perfectly complementary nucleic acid sequence. In some embodiments, the annealing step is combined with an extension step, and the extension step is performed at a temperature at which a polymerase enzyme in the reaction solution is active (e.g., between about 70° C. and 75° C.). In some embodiments, the annealing step is followed by an extension step that is performed at a temperature at which a polymerase enzyme in the reaction solution is active (e.g., between about 70° C. and 75° C.).

When multiple amplifications are being performed (for example, when two different regions are being analyzed simultaneously), LEL-PCR primers can be designed to accommodate amplification under the same reaction conditions with similar priming efficiencies.

### TI-PCR

In certain embodiments TI-PCR amplification methods are provided herein. TI-PCR is a PCR amplification method in which the temperature of the reaction vessel is elevated by heating at time-adjustable intervals for time-adjustable lengths of time and in which the temperature of the reaction vessel is decreased via passive cooling for time-adjustable intervals. The principles of TI-PCR can be applied to other PCR amplification techniques, including LATE-PCR and LEL-PCR.

Conventional PCR processes, in which all temperatures in a thermal cycle are carefully controlled, can be regarded as Temperature Precise PCR, regardless of whether the format of the reaction is symmetric, asymmetric, or LATE-PCR. Most devices designed for performing conventional PCR processes (e.g., thermocyclers) use electrical energy to precisely control heating and cooling in order to achieve the high and the low temperatures required at each step in a thermal cycle, as well as the length of time at each temperature (e.g., as depicted in FIG. 19).

As disclosed herein, in certain embodiments, TI-PCR significantly reduces the need to precisely control both the temperature used to melt nucleic acids strands and the lower temperature used to control the hybridization of strands, (e.g., as depicted in FIG. 19). In certain embodiments, TI-PCR includes increasing the temperature of the reaction vessel increased by heating the vessel at time-adjustable intervals and for time-adjustable lengths of time. The temperature of the reaction vessel is decreased via passive cooling for time-adjustable intervals. In some embodiments, active cooling is not used in at least half of the TI-PCR cycles. In some embodiments, active cooling is not used in any of the TI-PCR cycles. The length of the interval between the heat pulses determines the temperature to which the reaction falls during the passive cooling. In some embodiments, enzyme activity (e.g., its activity or inactivity at various temperatures) is regulated using temperature-dependent reagents that interact with the enzyme in a temperature dependent manner. In some embodiments, primers are used that have a relatively low melting temperature on the target nucleic acid sequence and a higher melting temperature on a perfectly complementary DNA sequence (e.g., a SuperSelective primer). These primers therefore hybridize to and extend on template strands at different temperatures at different points in the reaction. Examples of these properties of TI-PCR are illustrated in FIG. 19.

In some embodiments, TI-PCR can be performed using symmetric PCR, asymmetric PCR, or non-symmetric PCR (LATE-PCR) depending on initial concentrations and melting temperatures of the primers used. In some embodiments, TI-PCR can be performed using LEL-PCR.

In some embodiments, TI-PCR differs from conventional PCR in one or more of the following ways: 1) thermal cycling takes place in a device that only has the capacity to heat the sample; and 2) cooling at each step depends on the passive loss of heat. In some embodiments, the high temperature applied to the sample is not precisely controlled by the device other than by the length of time for which heat is applied. In some embodiments, the low temperature applied to the sample is not precisely controlled by the device other than by the length of time between heating cycles. In some embodiments, enzyme activity/specificity/and inactivity is determined by the presence of Temperature-Dependent Reagents that interact with the enzyme. Such reagents are described, for example, in the following patents and patent applications: U.S. Pat. No. 7,517,977; U.S. patent application publication No. 2012/0088275; and U.S. provisional patent application Nos. 61/755,872, 62/094,597 and 62/136,048, each of which are herein incorporated by reference in its entirety.

In some embodiments, because TI-PCR only depends on active heating, it is ideally suited for use in resource poor settings that have limited electric power. Moreover, in some embodiments, TI-PCR can be used under conditions in which the ambient temperature varies from run to run, or even during a run.

In some embodiments, devices that run TI-PCR reactions can be very simple in design. For example a reaction tube can be attached to a mechanism rotates the tube through a hot water bath and then through the air at varying rates, or a sample can be applied to a heating element that cycles between an on state and an off state at varying rates. In some embodiments, the passive dissipation of heat away from the reaction vessel is enhanced by making the volume of the sample small and/or by making the walls of the reaction chamber thin.

### SuperSelective Primers

In certain embodiments, SuperSelective primers are used in the methods described herein. SuperSelective primers have an anchor sequence, a bridge sequence, and a foot sequence that terminates in an extendable 3′ end. The anchor sequence can be of variable length, depending on the desired melting temperature to its target sequence. In some embodiments, the anchor is perfectly complementary to its designated initial target sequence. The bridge sequence is not complementary to the designated initial target sequence and, in some embodiments, has the fewest possible intra-molecular hybridization hairpins. In certain embodiments, the bridge sequence is generally between 14 and 45 nucleotides in length. In some embodiments, the bridge sequence can be adjusted as needed. For example, if several SuperSelective primers are used in the same reaction their bridge sequences are generally not the same. In some embodiments, the foot sequence is short, generally 5-8 nucleotides long. The foot sequence is perfectly complementary to a sequence within the designated initial target sequence that is some distance downstream, i.e. 3′, to the sequence that is complementary to the anchor of the same SuperSelective primer. In some embodiments, the foot is mis-matched to all allelic variants of the target sequence. Thus, a SuperSelective primer initially hybridizes to its designated target sequence by both the anchor sequences and the foot sequence. Under the same experimental conditions hybridization of the same SuperSelective primer to all allelic variants of the target sequence is less stable, because the foot portion of the primer is not fully complementary. Extension of the 3′end of a SuperSelective primer on its perfectly complementary target sequence is therefore more likely than extension on any allelic variant of the same target.

In some embodiments, a SuperSelective primer can be regarded to be the forward primer in a symmetric PCR amplification. In some embodiments, a second primer is used as the reverse primer in such a reaction. The target of the reverse primer lies downstream (3′) within the strand generated by extension of the SuperSelective primer, the Super-Select-Primer-Strand. During PCR amplification hybridization of the reverse primer to the Super-Select-Primer-Strand is followed by extension of the 3′ end of the reverse primer back to and through the entire length of the SuperSelective primer, thereby generating a Reverse Primer Strand that includes the complement of the foot, the bridge, and the anchor of the SuperSelective primer. The 5′ end of the Reverse Primer is fully complementary to the Super-Selective-Primer-Strand. In some embodiments, the Reverse Primer and SuperSelective primer are added to the Symmetric PCR reaction mixture at the same initial concentration, and used at a constant annealing temperature at which both primers hybridize to their respective target sequences.

In some embodiments, SuperSelective primers are used in LATE-PCR amplification. LATE-PCR utilizes a limiting primer and an excess primer which differ in their initial concentrations by at least 2-fold, 3-fold, 4-fold, 5-fold, 6-fold, 7-fold, 8-fold, 9-fold or 10-fold (e.g., at least 5-fold). In some embodiments, the initial concentration dependent melting temperatures of the limiting primer and the Excess primer adhere to the following equation Tm^{L}-Tm^{X}≧0. In some embodiments, the relationship between the melting temperature of the amplicon Tm^{A} and the melting temperature of the Excess primer is described by the equation Tm^{A}-Tm^{X}≦25° C.

In some embodiments, the SuperSelective primer serves as the limiting primer and have an initial concentration of about 50 nM. In some embodiments, the first round of amplification the Tm of the anchor to the designated target sequence is about 71° C. and in all subsequent rounds of replication the Tm of the SuperSelective primer to its full length complementary sequence is about 91° C.

In some embodiments, a SuperSelective primer is used in a LEL-PCR reaction. In some embodiments, the reverse primer serves as the Excess primer. In some embodiments, the Excess primer has extended non-complementary 5′ sequence that only hybridizes to the subsequent product of SuperSelective primer extension. Thus, in some embodiments, the initial Tm of the excess primer at an initial concentration of 1000 nM is 60° C., while in subsequent rounds of replication the Tm of the Excess primer to its full length complementary sequence is 81° C. In some embodiments, the initial melting temperatures of the amplicon (87° C.) and that of the excess primer (60° C.) fall outside of the bounds of LATE-PCR.

In some embodiments, the combined properties of the SuperSelective primer and the reverse primer allow for the introduction of novel temperature steps into the amplification protocol that facilitate greater stringency of hybridization between a primer and a designated target sequence and compared to its allelic variants. For example, in some embodiments, the one or more cycles of amplification can use an annealing temperature that is above the annealing temperature of the reverse primer for the target nucleic acid sequence (e.g., 70° C.)., which therefore results in one or more rounds of linear amplification of the Super-Selective-Primer-Strand only. In some embodiments, the annealing temperature can then be lowered for one or more cycles to a temperature below the melting temperature of the reverse primer on the target nucleic acid sequence in order to allow the 3′ target specific portion of the Excess primer to hybridizes to and extend along each previously generated Super-Selective-Primer Strands. In some embodiments, the annealing temperature can be raised again to 70-75° C., permitting hybridization and extension of the full length SuperSelective primer and the full length reverse primer.

In some embodiments, certain temperature sensitive amplification reagents are included in the reaction mixture. Such reagents are described, for example, in the following patents and patent applications: U.S. Pat. No. 7,517,977; U.S. patent application publication No. 2012/0088275; and U.S. provisional patent application No. 61/755,872, each of which are herein incorporated by reference in its entirety.

In some embodiments, the combination of primers and reagents described herein favor amplification of any designated target sequence over its allelic variants, when such variations lie in the sequence complementary to the foot of the SuperSelective primer.

### Optimization of LEL-PCR Amplification and Multiplexing

In some embodiments, primer sets are optimized for specificity and efficiency in monoplex LEL-PCR reactions using SYBR-Green before being integrated into the multiplex assay. In some embodiments the reproducibility and specificity of such reactions is enhanced by addition of one or more additive reagents (e.g., reagents described in U.S. Pat. No. 7,517,977, hereby incorporated by reference in its entirety) that increase polymerase selectivity and thereby suppress non-specific amplification and enhance multiplexing of primer pares (e.g., as described in Rice et al., Nat Protoc 2:2429-2438 (2007), hereby incorporated by reference in its entirety).

### Detection and Analysis of LEL-PCR Products

LEL-PCR makes it possible to generate relatively short or relatively long single-stranded amplicons which can then be scanned for sequence variations using, for example, one or more pairs of Lights-On/Lights-Off probes that are fluorescently labeled in one or more colors, Lights-Off Only probes in combination with a ds-DNA dye and/or by sequencing of amplification the amplification product.

Lights-On/Lights-Off probes are a pair of probes, as well as sets comprised of two or more pairs of probes that hybridize to adjacent nucleic acid sequences on a single-stranded DNA target, such as that produced by LEL-PCR amplification. The single-stranded DNA targets can include one or more targets generated in a LEL-PCR reaction. In some embodiments the informative sequence within each such target is hybridized to one or more pairs of Lights-On/Lights-Off probes. Each “Lights-On” probe is labeled with a fluorophore and a quencher and can be, for example, a molecular beacon with a self-complementary stem capable of base pairing for one or more contiguous complementary nucleotides. Each “Lights-Off” probe is labeled only with a quencher moiety that can absorb energy from the fluorophore of an adjacently hybridized “Lights-On” probe when both are bound to the target. In some embodiments use of Lights-On/Lights-Off probes allow for the detection of single nucleotide sequence differences by monitoring the effect of temperature changes on the fluorescence emissions of a probe/target mixture. Lights-On/Lights-Off probe technology is more fully described in PCT application No. PCT/US10/53569, hereby incorporated by reference in its entirety. The design criteria for pairs of Lights-On/Lights-Off probes are described in Rice et al., Nucleic Acids Research 40:e164 (2012) and PCT application No. PCT/US10/53569, each of which is incorporated by reference in its entirety.

In certain embodiments, sets of Lights-On/Lights-Off probes, (e.g., one for each amplicon), are designed to hybridize to the single-stranded amplicons at the end of a LEL-PCR amplification over the same wide temperature range, the temperature range being at least 5° C. below the limiting primer annealing temperature of the reaction. In some embodiments each set is labeled in a different color and each set spans the entire non-primer sequence of the amplicon. In certain embodiments, some probes include nucleotide mismatches to their target sequences to adjust the probe melting temperature. Lights-On probes are labeled with a fluorophore and a quencher at opposite ends. Lights-Off probes labeled with a quencher at the 5′ end are blocked at their 3′ end, for example, with a by covalent linkage of a three carbon, C3, moiety.

In some embodiments, the LEL-PCR amplification product is detected and analyzed using Lights-Off Only probes. Analysis of single-stranded amplification products using Lights-Off Only probes is similar to detection using Lights-On/Lights-Off probe sets, except a dsDNA fluorescent dye, such as SYBR® Green, is used in the place of a fluorescently labeled Lights-On probe. Thus, in certain embodiments, single-stranded DNA amplification products are detected using dyes that fluoresce when associated with double strands in combination with one or more hybridization probes that hybridize to a target nucleic acid sequence and that are labeled with a non-fluorescent quencher moiety, for example, a Black Hole quencher (“Lights-Off Only probes”). The fluorescent signature produced by from the dsDNA binding dye as a function of temperature over a temperature range that includes the melting temperature of such hybridization probe or probes is analyzed to detect sequence variations indicating the original methylation state of the target sequence.

In some embodiments the Lights-On probes and/or the Lights-Off probes are designed taking into account constraints imposed by the target sequences and temperature dependent secondary structures of the single-stranded target amplicons. For instance, one or two contiguous probes may bind to a sequence at a temperature that is higher than that needed for the sequence to form a hairpin loop, thereby preventing loop formation when the temperature is lowered. Other information with regard to the design of Lights-On/Lights-Off probes are described, for example, in Rice et al., Nucleic Acids Res (2012) and Carver-Brown et al., J Pathog 2012:424808 (2012), each of which is hereby incorporated by reference in its entirety.

In some embodiments multiplex LEL-PCR amplification is carried out according to standard LATE-PCR conditions (e.g., 25 μl reactions consisting of 1× Platinum Taq buffer, 3 mM MgCl2, 400 μM of each deoxynucleotide triphosphate, 50 nM of each limiting primer, 1 μM of each excess primer, 100 nM of each Lights-On probe, 300 nM of each Lights-Off probe, 2.0 units of Platinum Taq DNA polymerase, and bisulfite-treated genomic DNA). In some embodiments the concentration of each Lights-On probe is slightly less than the anticipated maximal yield of single-stranded DNA amplicons generated in the LEL-PCR to guarantee complete binding of all the Lights-On probes and minimize differences among replicate fluorescent signatures (e.g., 100 nM if the above amplification conditions are used). In some embodiments the concentration of each Lights-Off probe is set three-fold higher than the concentration of each Lights-On probe to guarantee that every bound Lights-On probe will have a Lights-Off probe hybridized next to it at low temperature (e.g., 300 nM if the above conditions are used). In some embodiments amplification is carried out for at least 30, 35, 40, 45, 50, 55, 60, 65, 70, 75 or 80 cycles.

In some embodiments at least one non-amplifiable pair of oligonucleotides, comprised of a fluorescently labeled oligonucleotide and a complementary oligonucleotide labeled with a moiety that quenches fluorescence (such as a Black Hole Quencher) is added to the amplification reaction. The melting temperature of the non-amplifiable pair of oligonucleotides is higher than the melting temperature of all probe-target hybrids in the reaction. The pair of non-amplifiable oligonucleotides that serves as an internal temperature mark may also serve to enhance polymerase selectivity as described in U.S. Provisional Pat. App. No. 61/755,872, hereby incorporated by reference in its entirety.

In certain aspects, a Temperature Dependent Reagent is included in the amplification reaction. In some embodiments, the Temperature Dependent Reagents described herein reduce or prevent Type 1 and/or Type 2 mispriming. In some embodiments, the Temperature Dependent Reagents reduce or prevent the formation of non-specific products during reverse transcription reactions. In some embodiments, the Temperature Dependent Reagents provided herein reversibly acquires a principally stem-loop hairpin conformation at a first temperature but not at a second, higher temperature. In some embodiments, the first temperature is a temperature that is below an annealing temperature of an amplification reaction and the second temperature is a temperature that is above the annealing temperature of an amplification reaction. In certain embodiments, the stem-loop hairpin confirmation of the Temperature Dependent Reagent inhibits the activity and/or increases the specificity of a thermostable DNA polymerase (e.g., Taq polymerase) and or a reverse transcriptase. In some embodiments, the mispriming prevention region comprises non-identical moieties attached to its 5′ and 3′ termini (not including linkers, if present). In some embodiments, the terminal moieties are cyclic or polycyclic planar moieties that do not have a bulky portion (not including the linker, if present), such as a dabcyl moiety, a Black Hole Quencher moiety (e.g., a Black Hole Quencher 3 moiety) or a coumarin moiety (e.g., coumarin 39, coumarin 47 or Biosearch Blue). In some embodiments, the Temperature Dependent Reagents contains a loop nucleic acid sequence made up of a single nucleotide repeat sequence (e.g., a poly-cytosine repeat). Thus, in some embodiments, the Temperature Dependent Reagents is able to act as both a “hot-start” reagent and a “cold-stop” reagent during the performance of a primer-based nucleic acid amplification process. Certain embodiments of the single-stranded Temperature Dependent Reagents described herein are referred to as ThermaStop reagents.

In certain aspects, used herein is a multi-stranded Temperature Dependent Reagent comprising at least two non-identical 5′ or 3′ terminal moieties (not including linkers, if present). In some embodiments, the multi-stranded Temperature Dependent Reagent inhibits or prevents Type 3 and/or Type 4 mispriming. In some embodiments, the multi-stranded Temperature Dependent Reagent comprises a first nucleic acid strand of and a second nucleic acid strand that collectively comprise at least two non-identical 5′ or 3′ terminal moieties. In some embodiments, the at least two non-identical moieties are selected from dabcyl moieties, Black Hole Quencher moieties (e.g., Black Hole Quencher 3 moieties) and coumarin moieties (e.g., coumarin 39, coumarin 47 and Biosearch Blue). Certain embodiments of the single-stranded Temperature Dependent Reagents described herein are referred to as ThermaGo reagents.

In some embodiments target amplification, and product analysis are carried out in a single-tube. Target amplification and analysis can take place as a closed-tube homogeneous LEL-PCR amplification reaction followed by end-point analysis of the single-stranded DNA product using, for example, either Lights-On/Lights-Off probes or Lights-Off only probes to analyze the nucleotide composition of the target.

In addition, in some embodiments blockers are used that bind to their targets in an allele specific manner and selectively prevent primer extension. Some blockers, such as those made of LNA's and PNA's are located downstream of the 3′ end of the primer. Other blockers overlap with the 3′ end of the primer. Such approaches can be adapted for use with LEL-PCR by designing the limiting primer to be a selective primer and the excess primer to be a non-selective primer.

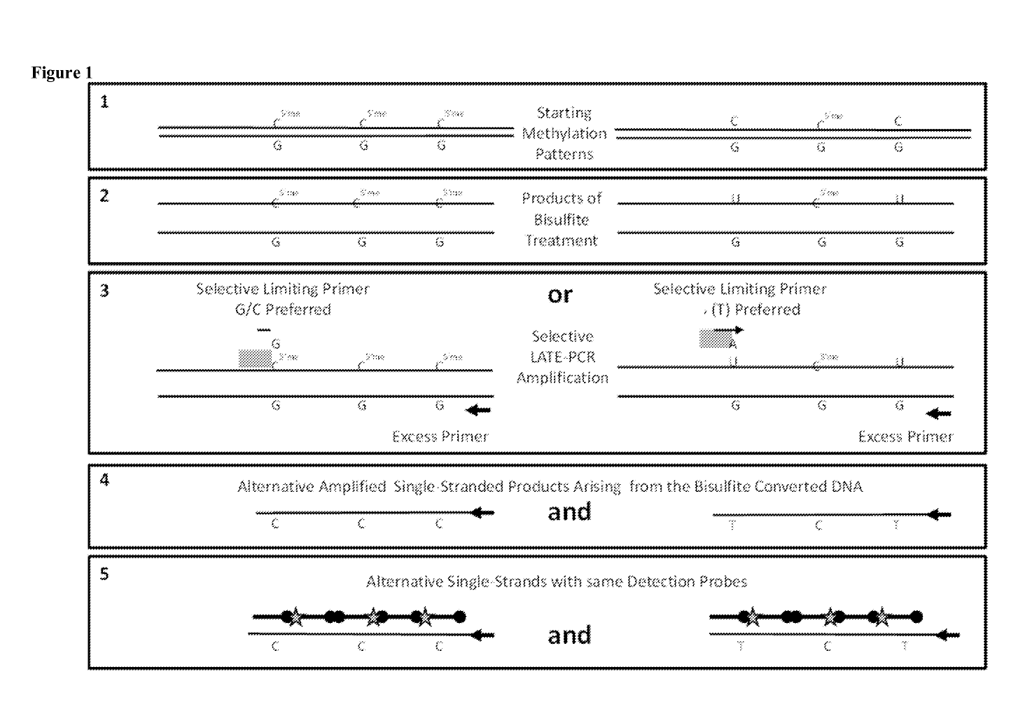

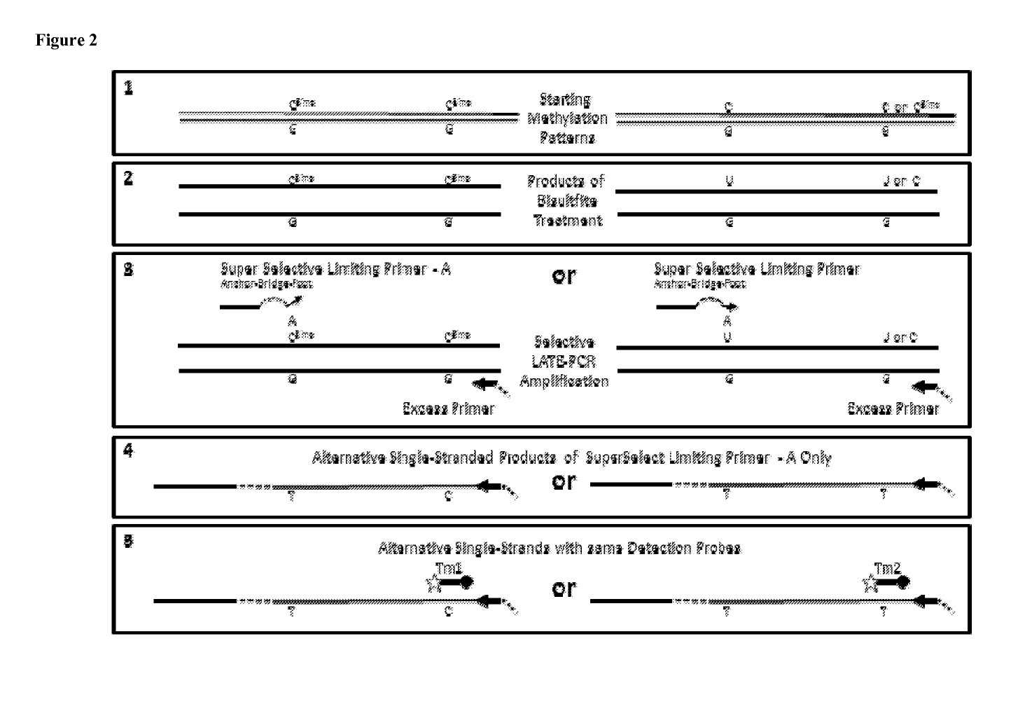

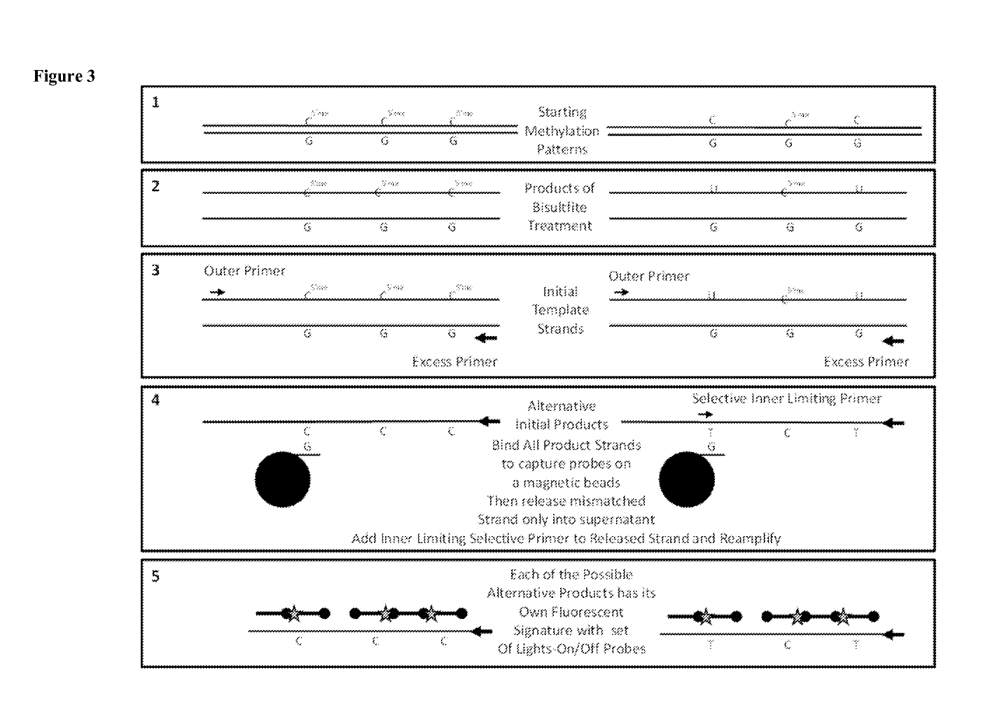

As one skilled in the art will appreciate the selective primer approach described above and in FIG. 1 can be combined with the selective magnetic bead approach above and in FIG. 3. In one instance the reaction can begin as described in FIG. 3 but can proceed as described in FIG. 1 but making the Inner limiting primer into a selective primer. In another instance, the reaction can begin as described in FIG. 1 with preferential amplification of one sequence variant, followed by magnetic bead removal of the undesired variant or variants, followed by re-amplification of the desired variant using an Inner limiting primer.

### EXAMPLES

### Example 1—Allelic-Discrimination Using SuperSelective Primers

SuperSelective primers and their corresponding reverse primers were used to illustrate in two separate PCR assays the preferential amplification of target sequences in which the 3′ end of the SuperSelective primers formed fully complementary hybrids (matched targets) versus target sequences in which the 3′ end of the SuperSelective primers formed mismatched hybrids (mismatched targets). The first PCR assay selectively amplified the single nucleotide L858R mutation within the human epidermal growth factor receptor (EGFR) gene that confers sensitivity to the tyrosine kinase inhibitors such as gefitinib. The second PCR assay selectively amplified the single nucleotide V600E mutation within the human B-RAF gene that confers sensitivity to the tyrosine kinase inhibitor vemurafenib.

The sequence of the SuperSelective primers and their corresponding reverse primers for the two PCR assays were as follows:

### Forward EGFR SuperSelective Primer Complementary to the L858R Mutation:

[SEQ ID NO: 1]
5′ CTGGTGAAAACACCGCAGCATGTCGCACGAGTGAGCCCTGGGCGG 3′

where the sequence preceding the underlined sequence corresponds to the anchor sequence, the underlined sequence corresponds to the bridge sequence, the sequence following the underlined sequence corresponds to the foot sequence, and the nucleotide shown in bold corresponds to the nucleotide that is matched the to the EGFR L858R mutation and mismatched to the wild-type sequence. The melting temperature of the EGFR anchor sequence is 71° C.

### Reverse EGFR Primer:

[SEQ ID NO: 2]
5′ GCATGGTATTCTTTCTCTTCCGCA 3′

which has a Tm of 60° C.
Forward BRAF SuperSelective primer complementary to the L858R mutation:

[SEQ ID NO: 3]
5′ AGACAACTGTTCAAACTGATGGGAAAACACAATCATCTATTTCTC 3′

where the sequence preceding the underlined sequence corresponds to the anchor sequence, the underlined sequence corresponds to the bridge sequence, the sequence following the underlined sequence corresponds to the foot sequence, and the nucleotide shown in bold corresponds to the nucleotide that is matched the to the BRAF V600E mutation and mismatched to the wild-type sequence. The melting temperature of the BRAF anchor sequence is 71° C.

### Reverse B-RAF Primer:

[SEQ ID NO: 4]
5′ AGACAACTGTTCAAACTGATGGGA 3′

which has a Tm of 60° C.

The DNA targets were located on plasmids containing in which a 115-base pair gene fragment from EGFR exon 21 containing the L858R mutant sequence, the corresponding EGFR wild-type sequence, a 116-base pair fragment B-RAF V600E mutant sequence, or the corresponding B-RAF wild-type sequence, were inserted into a pGEM-11Zf(+) plasmid. These plasmids were digested with the endonuclease MseI (New England Biolabs). Prior to the use with SuperSelective primers, corresponding pairs of mutant and wildtype targets were matched in concentration to 10,000 copies/μl each by dilution in 10 mM Tris-Cl, pH 8.3. Equimolar primer concentrations were confirmed in separate reactions after real-time amplification with SYBR Green using the following primers that do not overlap with the mutations together with their corresponding reverse primer:

### EGFR Forward Anchor:

[SEQ ID NO: 5]
5′ CTGGTGAAAACACCGCAGCATGTC 3′

### BRAF Forward Anchor:

[SEQ ID NO: 6]
5′ AGACAACTGTTCAAACTGATGGGA 3′

Amplifications were carried out in replicate reactions in a Stratagene MX3005P thermal cycler (Agilent, Santa Clara, Calif.). PCR assays were performed in a 30 μl volume containing of 1× Platinum Taq buffer (Invitrogen, Carlsbad, Calif.), 3 mM MgCl2, 250 μM of each deoxynucleotide triphosphate, 60 nM of forward SuperSelective primer, 60 nM of reverse primer, 0.24×SYBR Green (Invitrogen, Carlsbad, Calif.), 1.5 units of Platinum Taq DNA polymerase (Invitrogen, Carlsbad, Calif.), and 10,000 copies of either mutant or wild-type plasmids. The reactions were first incubated at 95° C. for three minutes, followed by 60 cycles of denaturation at 95° C. for 15 seconds, primer annealing at 60° C. for 15 seconds, and primer extension at 72° C. for 30 seconds. SYBR Green fluorescent intensity was measured during each extension step throughout the course of each reaction.

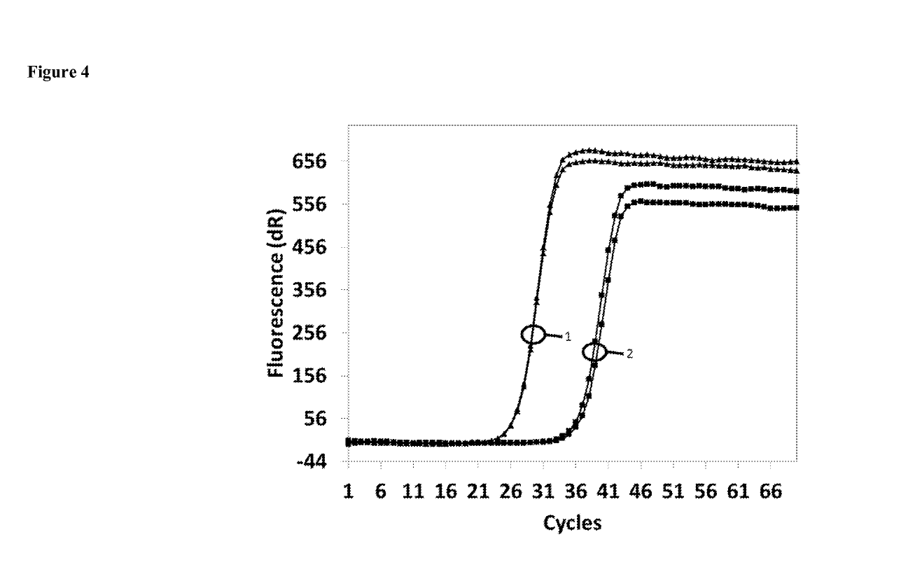

FIG. 4 shows preferential amplification of 10,000 copies of mutant EGFR L858R DNA (Curve 1) relative to 10,000 copies of wild-type EGFR DNA (Curve 2).

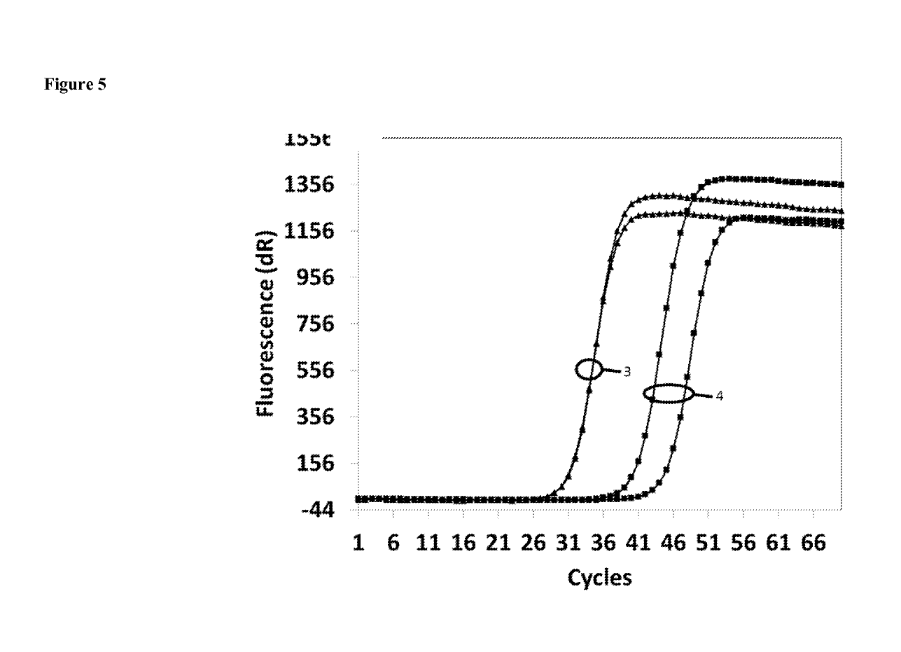

FIG. 5 shows preferential amplification of 10,000 copies of mutant BRAF V600E DNA (Curve 3) relative to 10,000 copies of wild-type BRAF DNA (Curve 4).

### Example 2—Increasing Concentrations of Temperature-Dependent Reagent EP003 Enhances Allele Discrimination of SuperSelective Primers

Temperature-Dependent Reagents is a category of terminally-modified, double-stranded DNA additives that, depending on the configuration of the modifications at the end of the strands and the concentration of the reagent, determine the selectivity/specificity and/or activity of Taq DNA polymerase in PCR amplification reactions in a temperature-dependent manner (e.g., as described in U.S. patent application publication No. 2012/0088275, which is hereby incorporated by reference). The temperature dependency is due to fact that Temperature-Dependent Reagent is only active at temperatures where it remains double-stranded. This class of reagents make it possible to define the stringency of the reaction not only by controlling the temperature of the assay in a precise manner, as in conventional PCR, but also by allowing the reaction to cool down to within a range of temperatures where the reagent becomes double stranded.

As described herein, Temperature-Dependent Reagent EP003 improves the selectivity of SuperSelective primers without altering the temperature of the annealing or extension steps of the reaction. The composition of double stranded Temperature-Dependent Reagent EP003 is as follows:

[SEQ ID NO: 7]
5′ GGAGCAAAATAGCAATGAGGTA 3′ [Dabcyl-Q]
[SEQ ID NO: 8]
3′ [Dabcyl-Q]CCT CGTTTT ATC GTTACT CCAT [5-
Dabcyl]5′

The resulting EP003 hybrid has a melting temperature of 63.1° C. at a concentration of 100 nM.

The same amplification reactions described in Example 1 were carried out in the presence of increasing concentrations of EP003 (0 nM, 25 nM, 50 nM and 100 nM). Amplifications were done in replicate reactions in a Stratagene MX3005P thermal cycler (Agilent, Santa Clara, Calif.). PCR assays were performed in a 30 μl volume containing 1× Platinum Taq buffer (Invitrogen, Carlsbad, Calif.), 3 mM MgCl2, 250 μM of each deoxynucleotide triphosphate, 60 nM of forward SuperSelective primer, 60 nM for reverse primer, 0.24×SYBR Green (Invitrogen, Carlsbad, Calif.), 0-100 nM EP003, 1.5 units of Platinum Taq DNA polymerase (Invitrogen, Carlsbad, Calif.), and 10,000 copies of either mutant or wild-type plasmids. The reactions were first incubated at 95° C. for three minutes, followed by 60 cycles of denaturation at 95° C. for 15 seconds, primer annealing at 60° C. for 15 seconds, and primer extension at 72° C. for 30 seconds. SYBR Green fluorescent intensity was measured during each extension step throughout the course of each reaction.

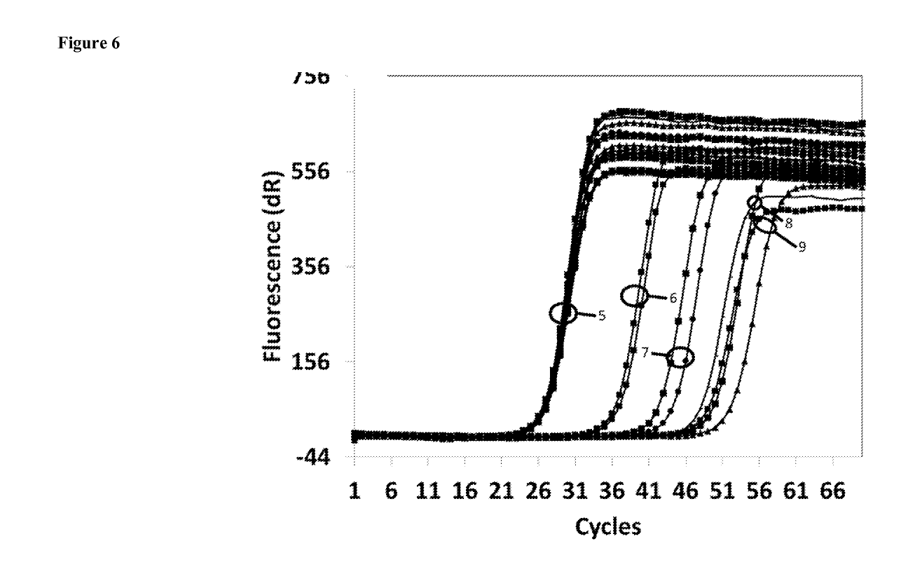

FIG. 6 shows that increasing concentrations of EP003 did not appreciably affect amplification of EGFR L858R mutant targets (Curve 5, all EP003 concentrations) but preferentially delayed the amplification of wild-type EGFR targets (Curve 6, 0 nM EP003; Curve 7, 25 nM EP003; Curve 8, 50 nM EP003; Curve 9, 100 nM EP003).

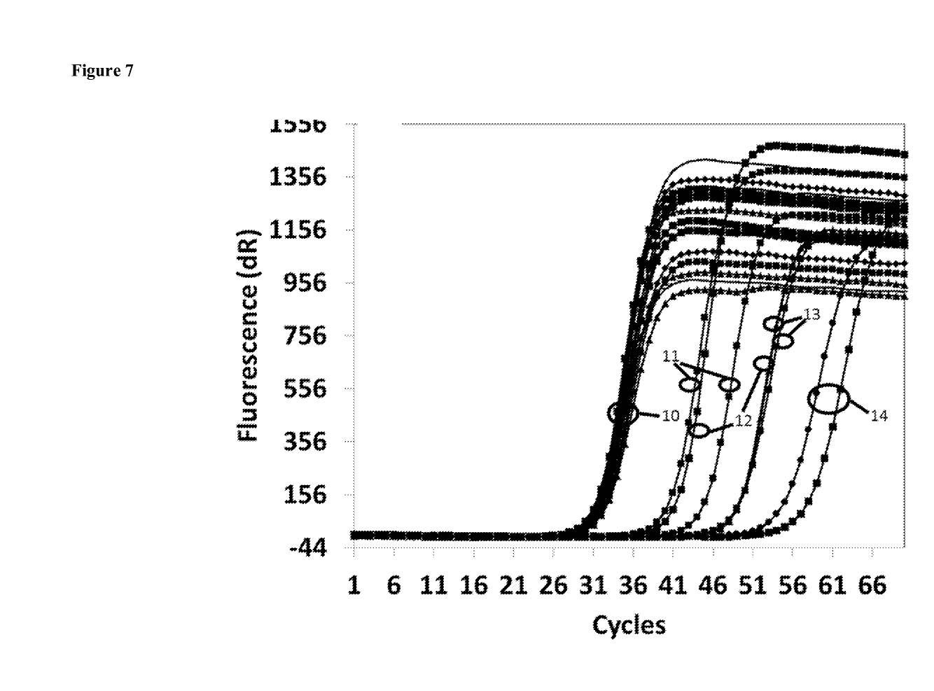

FIG. 7 shows that increasing concentrations of EP003 did not appreciably affect amplification of BRAF V600E mutant targets (Curve 10, all EP003 concentrations) but preferentially delayed the amplification of wild-type BRAF targets (Curve 11, 0 nM EP003; Curve 12, 25 nM EP003; Curve 13, 50 nM EP003; Curve 14, 100 nM EP003).

Temperature-Dependent Reagent EP003 can be combined with other Temperature-Dependent Reagents with various configurations of terminal modifications (e.g., as used in Example 11 or as described in U.S. Patent application publication No. 2012/0088275 and U.S. provisional patent application No. 61/755,872) to further improve primer specificity/selectivity.

### Example 3—SuperSelective Primers in LATE-PCR and LEL-PCR Reactions

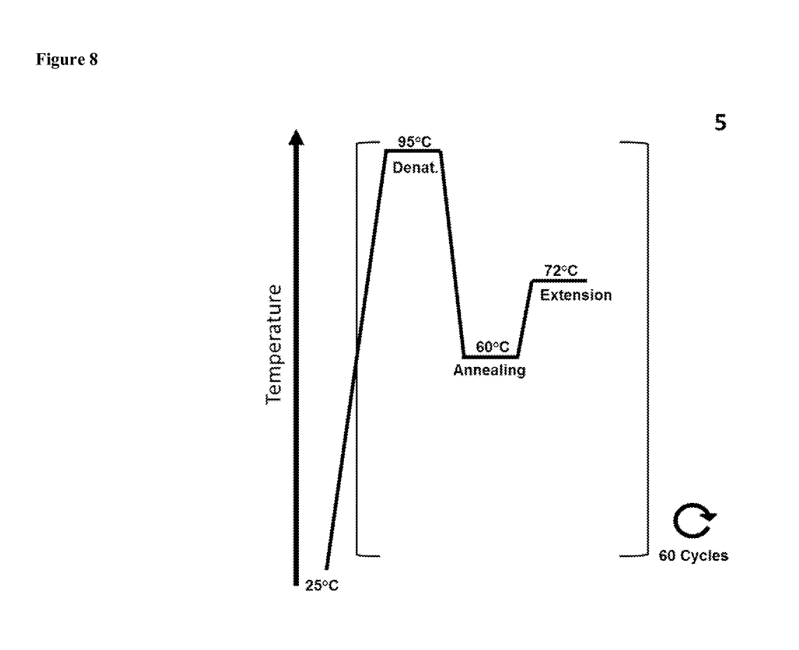

Mutant templates are distinguished from wild-type templates when SuperSelective primers hybridize to target molecules in the original sample. When SuperSelective primers overlap the sequence difference between mutant and wild-type, amplicons resulting from mispriming on wild-type targets are identical to amplicons from mutant targets. It would therefore be desirable to restrict the number of thermal cycles in which the SuperSelective primers hybridize to the original wild-type targets to minimize the possibility of unintended initiation events on wild-type targets. It would also be desirable to increase the stringency of hybridization of the SuperSelective primers in order to minimize unintended extension on wild-type targets. The temperature cycling profile used in conventional SuperSelective primer based PCR does not provide such flexibility (i.e., since the melting temperatures of the anchor sequence of the SuperSelective primer and the reverse primers typically are 71° C. and 60° C., respectively, any attempt to increase the stringency of the SuperSelective primers by raising the annealing temperature above 60° C. would reduce the number of reverse primers participating in the reaction and result in lower amplification efficiency, FIG. 8).

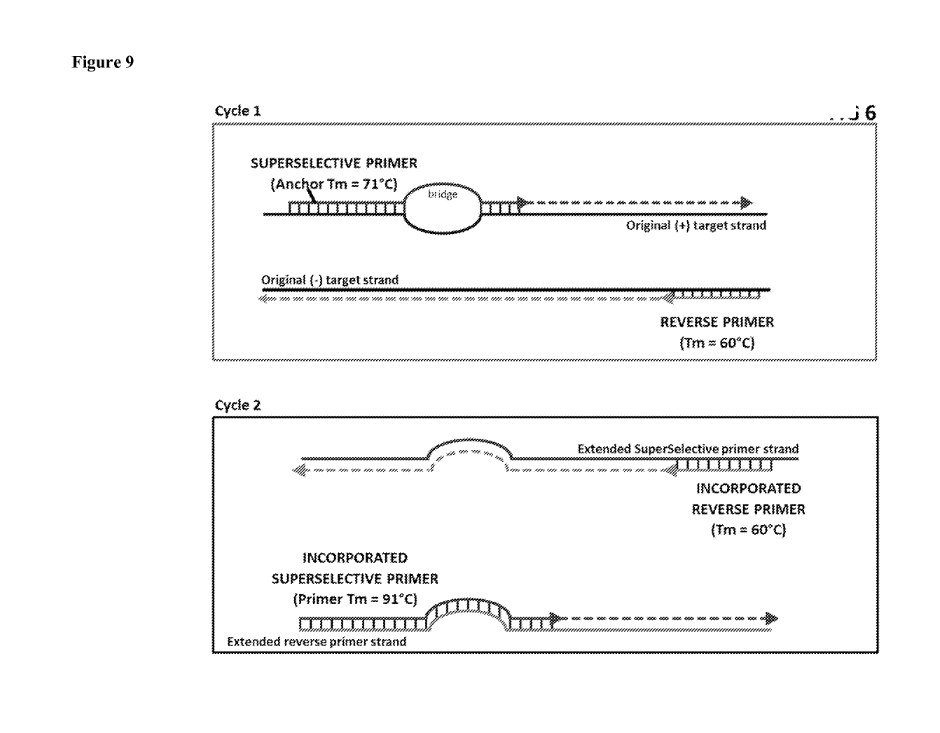

A feature of certain SuperSelective primers is that the 5′-anchor sequence is linked to the 3′-foot sequence by a 14-nucleotide long bridge sequence that is not complementary to the target sequence. As a result, when the primer is hybridized to an original target molecule, the bridge sequence in the primer remains single stranded and forms a bubble. Once a SuperSelective primers are successfully extended on the matched target sequence, however, the amplicons produced in subsequent amplification cycles incorporate the entire SuperSelective primer sequence (including the bridge sequence) and the melting temperature of the SuperSelective primer on the now fully complementary amplicon increases (FIG. 9).

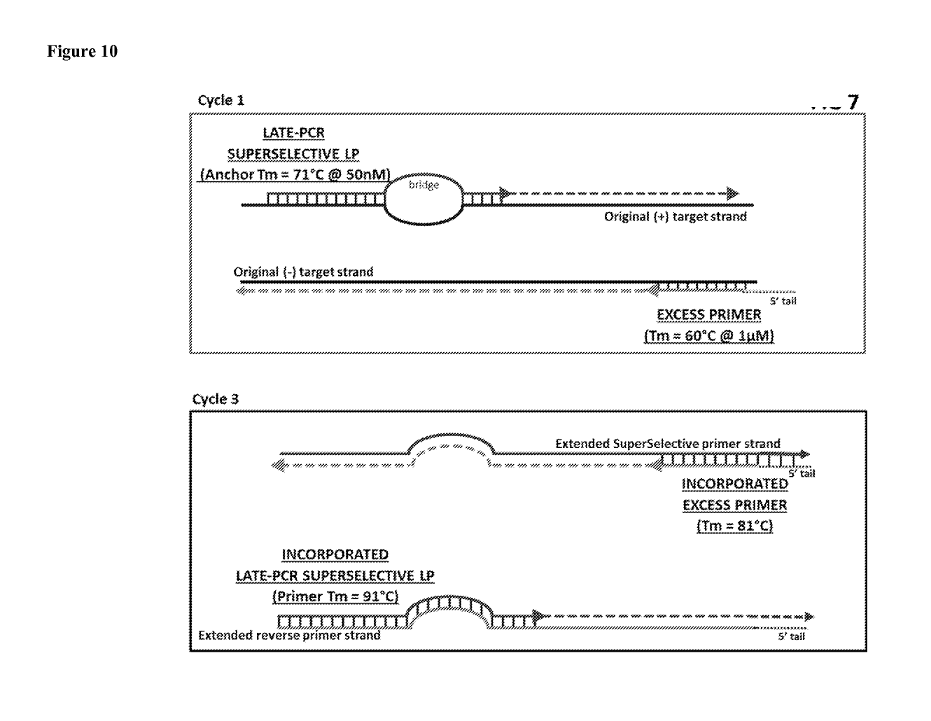

By designing the reverse primer such that its Tm also increases during amplification, it is possible to restrict the number of thermal cycles where the SuperSelective primers hybridize to the original target nucleic acid sequence by raising the annealing temperature of the reaction to a temperature where only fully complementary primers bound to amplicon targets participate in the reaction. To accomplish this, the SuperSelective primer and the reverse primer were converted to LATE-PCR primers and a 5′ tail non-complementary to the original target sequence was added to the LATE-PCR reverse primer such that once incorporated into an amplicon, the Tm of the fully complementary reverse primer on the amplicons targets increased after the first cycle of amplification (FIG. 10).

To convert conventional primer pairs to LATE-PCR primers, the SuperSelective primer was used as the limiting primer at 50 nM without any modification. The length of the reverse primer including addition of a 5′ tail non-complementary to the original target sequence was adjusted to allow this primer to be used as an excess primer at 1000 nM while keeping the concentration-adjusted Tm at 60° C.

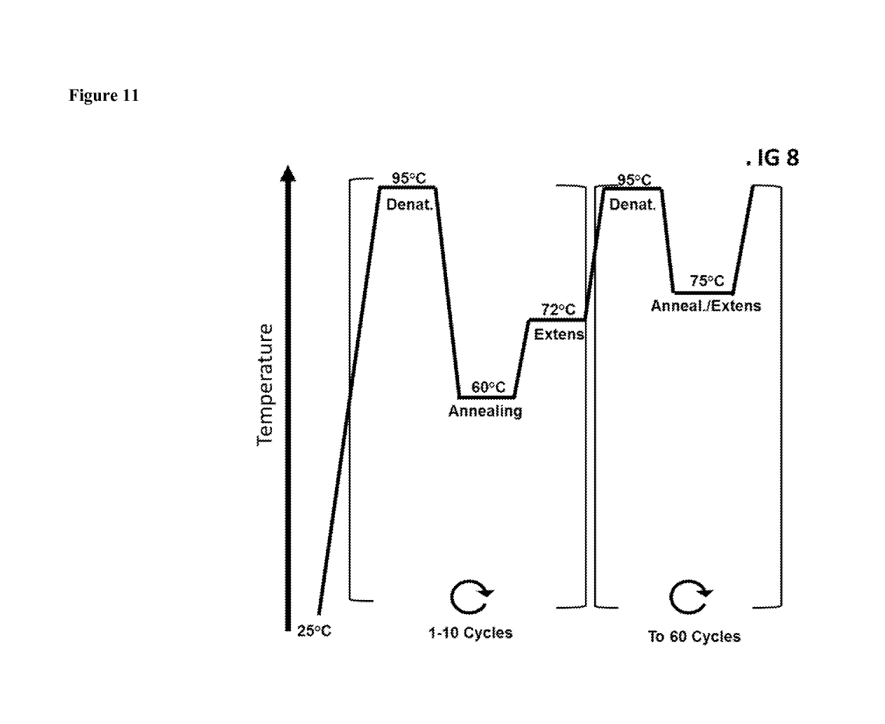

The LATE-PCR primer design allows a different temperature cycling profile to be used with SuperSelective primers (e.g., as depicted in FIG. 11). In this scheme, the original target molecules are interrogated with SuperSelective primers for selective amplification of mutant targets in the first 1-10 amplification cycles. During these initial cycles, the excess primers do not meet LATE-PCR design criteria, since the Tm of these primers (60° C.) is more than 20° C. below the amplicon Tm (LATE-PCR design criteria generally specifies that the excess primer Tm should be less than 20° C. the amplicon Tm). After these initial selective cycles, the annealing temperature is increased to 75 C to allow exclusive exponential amplification of amplicon targets without any further interrogation of original target molecules.

By design, the Tm of the anchor sequence of the SuperSelective primer is >10° C. above the excess primer Tm (71° C. compared to 60° C.). This allows the implementation of a PCR approach wherein the SuperSelective primer undergoes several rounds of linear amplification at an annealing temperature of 71° C. to interrogate mutant and wild-type targets under stringent conditions. The temperature is then dropped once to 60° C. to enable hybridization and extension of the excess primer for one cycle. This is followed by multiple rounds of temperature cycles with an annealing temperature of 75° C. to enable exclusive amplification of amplicon targets by LATE-PCR. This approach where linear amplification of a primer is followed by exponential amplification and linear amplification under LATE-PCR conditions is called Linear-Exponential-Linear (LEL) PCR (FIG. 12).

To show proof-of-principle of LEL-PCR, the following LATE-PCR SuperSelective primer and reverse excess primers were used:

### LATE-PCR SuperSelective Limiting Primer:

[SEQ ID NO: 9]
5′ CTGGTGAAAACACCGCAGCATGTCGCCCGAGTGAGCCCTGGGCAG
3′

The sequence preceding the underlined sequence corresponds to the anchor sequence (24 nucleotides), the underlined sequence corresponds to the bridge sequence, the sequence following the underlined sequence corresponds to the foot sequence, and the nucleotide shown in bold corresponds to the nucleotide that is matched the a T nucleotide on the matched original target sequence. The concentration-adjusted Tm of the anchor sequence is 71° C. at 50 nM.

LATE-PCR Excess Reverse Primer with a 5′ Non-Complementary Tail (Underlined):

[SEQ ID NO: 10]
5′ GAGGAATGAAACGGAGGAAGACGTACGTATTCTTTCTCTTCCGCA
3′

The underlined sequence corresponds to the 5′ tail non-complementary to the original target sequence. The concentration-adjusted Tm of the this primer is 60° C. at 1000 nM.

The original targets for these primers consisted of the following double-stranded synthetic oligonucleotides (IDT, Coralville, Iowa):

### Matched Target:

[SEQ ID NO: 11]
5′CTAAAGCCACCTCCTTACTTTGCCTCCTTCTGCATGGTATTCTTTCTC
TTCCGCATGCACCAGTTTGGCCTGCCCAAAATCTGTGATCTTGACATGCT
GCGGTGTTTTCACCAGTACGTTCCTGG3′

The foot region of the LATE-PCR SuperSelective primer is fully complementary at the T nucleotide, shown in bold.

### Mismatched Target:

[SEQ ID NO: 12]
5′CTAAAGCCACCTCCTTACTTTGCCTCCTTCTGCATGGTATTCTTTCTC
TTCCGCATGCACCAGTTCGGCCCGCCCAAAATCTGTGATCTTGACATGCT
GCGGTGTTTTCACCAGTACGTTCCTGG3′

The foot region of the LATE-PCR SuperSelective primer is mismatched at the C nucleotide.

Amplifications were carried out in replicate reactions in a Stratagene MX3005P thermal cycler (Agilent, Santa Clara, Calif.). PCR assays were performed in a 15 μl volume consisting of 1× Platinum Taq buffer (Invitrogen, Carlsbad, Calif.), 3 mM MgCl2, 250 μM of each deoxynucleotide triphosphate, 50 nM of LATE-PCR SuperSelective primer, 1000 nM for LATE-PCR reverse primer, 0.24×SYBR Green (Invitrogen, Carlsbad, Calif.), 1.5 units of Platinum Taq DNA polymerase (Invitrogen, Carlsbad, Calif.), and 10,000 copies of either matched un-methylated target or matched methylated target. The reactions were first incubated at 95° C. for three minutes, followed by 1-10 cycles of denaturation at 95° C. for 15 seconds and primer annealing at 70° C. for 15 seconds, and primer extension at 80° C. for 30 seconds (note: primer extension was done at 80° C. instead of the customary 72° C.-75° C. to prevent further hybridization events of the SuperSelective primer during extension), one cycle of denaturation at 95° C. for 15 seconds and primer annealing at 60° C. for 15 seconds, and primer extension at 72° C. for 30 seconds, and 50 cycles of 95° C. for 15 seconds and primer annealing/extension at 75° C. for 30 seconds. SYBR Green fluorescent intensity was measured during each extension step throughout the course of each reaction.

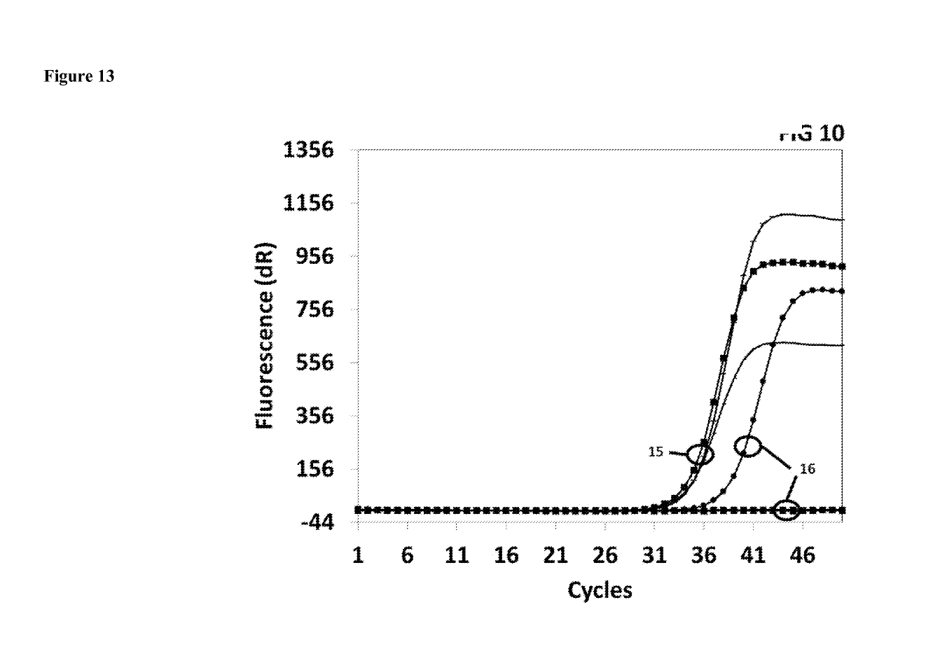

FIG. 13 shows preferential amplification of three replicates of 10,000 copies of matched targets (Curves 15) relative to only one out of three replicates of 10,000 copies of mismatched targets (Curves 16) after a single round of linear extension of the limiting SuperSelective primer at 70° C. This result demonstrates that it is possible to preferentially restrict the number of hybridization events occurring on mismatched target but at the expense of picking only a limited number of matched targets.

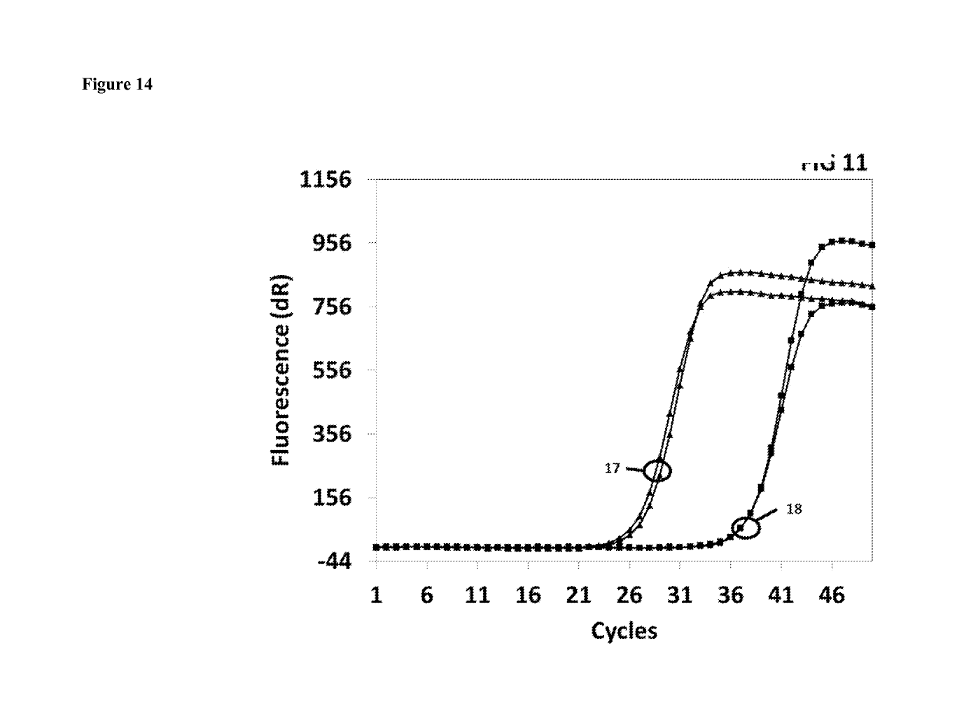

FIG. 14 shows that increasing the number of linear amplification cycles for the LATE-PCR SuperSelective limiting primer from one to ten allows better amplification of the matched targets (Curves 17) but enough mismatched targets hybridize under these conditions to allow amplification of all three replicates (Curves 18). These experiments demonstrate that LEL PCR allows for more stringent amplification of SuperSelective primers and fewer number of cycles where the SuperSelective primers interrogate the mismatched targets but that these changes do not improve the amplification selectivity. These experiments also demonstrate that SuperSelective primers can be targeted to sequence differences in a pair of targets.

### Example 4—LATE-PCR SuperSelective Primers in Combination with a Temperature-Dependent Reagent

The experiments in Example 3 were performed in the presence of another version of a Temperature-Dependent Reagent (Reagent 2, a double-stranded DNA with terminal modifications that include a fluorophore and a quencher, described in U.S. Patent application publication 2012/0088275) to test for improvements in selectivity.

### Fluorescent Temperature Dependent Reagent 2:

[SEQ ID NO: 13]
5′ QSR670-CAGCTGCACTGGGAAGGGTGCAGTCTGACC-C3 3′
[SEQ ID NO: 14]
5′ GGTCAGACTGCACCCTTCCCAGTGCAGCTG-BHQ2 3′

Amplifications were carried out in replicate reactions in a Stratagene MX3005P thermal cycler (Agilent, Santa Clara, Calif.). PCR assays were performed in a 15 μl volume consisting of 1× Platinum Taq buffer (Invitrogen, Carlsbad, Calif.), 3 mM MgCl2, 250 μM of each deoxynucleotide triphosphate, 50 nM of LATE-PCR SuperSelective primer, 1000 nM for LATE-PCR reverse primer, 0.24×SYBR Green (Invitrogen, Carlsbad, Calif.), 1.5 units of Platinum Taq DNA polymerase (Invitrogen, Carlsbad, Calif.), 25 nM Reagent 2 and 10,000 copies of either matched un-methylated target or matched methylated target. The reactions were first incubated at 95° C. for three minutes, followed by 10 cycles of denaturation at 95° C. for 15 seconds and primer annealing at 70° C. for 15 seconds, and primer extension at 80° C. for 30 seconds (note: primer extension was done at 80° C. instead of the customary 72° C.-75° C. to prevent further hybridization events of the SuperSelective primer during extension), one cycle of denaturation at 95° C. for 15 seconds and primer annealing at 60° C. for 15 seconds, and primer extension at 72° C. for 30 seconds, 50 cycles of 95° C. for 15 seconds and primer annealing/extension at 75° C. for 30 seconds, and a melting from 25° C. to 95° C. with fluorescent acquisition in the Cal Orange channel (to monitor the fluorescence of Reagent 2). SYBR Green fluorescent intensity was measured during each extension step throughout the course of each reaction to follow the amplification reaction in real time..

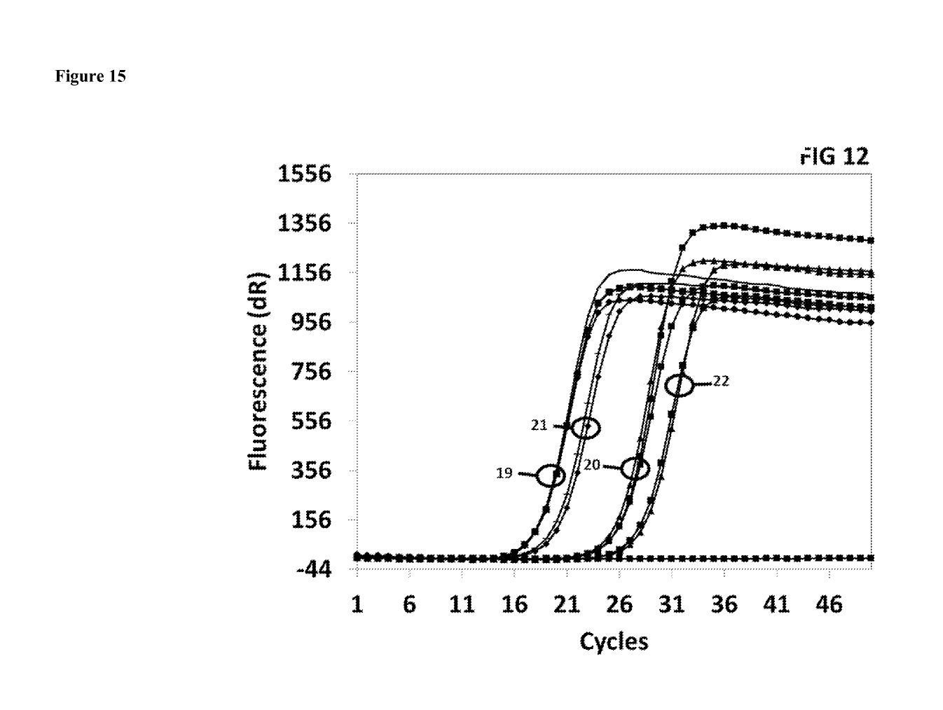

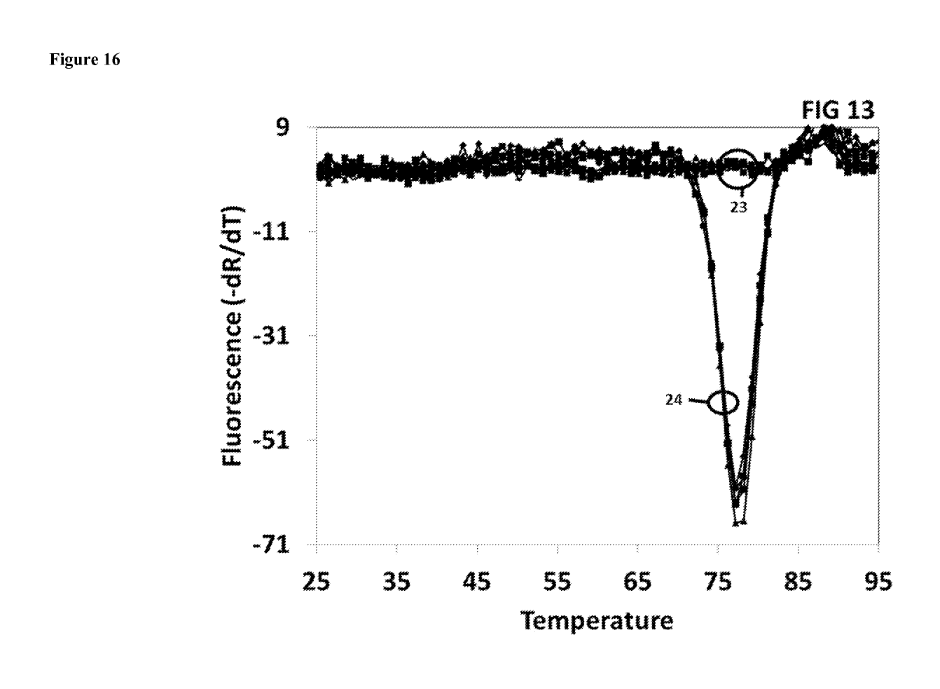

FIG. 15 shows that addition of 25 nM of the Reagent 2 increased the selectivity of the LATE-PCR SuperSelective primers by 0.8 Ct values. The delta Ct value between the matched target (Curves 19) and the mismatched target (Curves 20) was 7.3 cycles compared to the delta Ct value between the matched target+25 nM Reagent 2 (Curves 21) and the mismatched target+25 nM Reagent 2 (Curves 22).

Reagent 2 can be optimized further to achieve improved selectivity similar to Temperature-Dependent Reagent EP003, as described in provisional patent application No. 61/755,872, which is hereby incorporated by reference. Reagent 2 can be combined with other versions of Temperature Dependent Reagents to achieve even more improvements in selectivity, as described in U.S. patent application Ser. No. 13/256,038, which is hereby incorporated by reference.

These results, along with those in Example 2, demonstrate that Temperature Dependent Reagents can be used instead of precise temperature regulation to control the specificity of LATE-PCR SuperSelective primers. These examples demonstrate that LATE-PCR SuperSelective primers can be optimized by adjusting thermal cycles and by adjusting the type and combinations of Temperature Dependent Reagents such as EP003 and Reagent 2. Further possible optimizations to achieve maximal allele discrimination with SuperSelective primer include adjusting length of the foot, the length and melting temperature of the bridge sequence, the melting temperature of the anchor sequence of the SuperSelective primers themselves. Since the selective steps for differential amplification take place in the in the very early cycles, multiplexing with different pairs of SuperSelective limiting primers and reverse excess primers will reflect the number of starting copies of different alleles are present in the initial sample.

FIG. 16 shows that Reagent 2 present in the samples from FIG. 15 can be readily visualized by virtue of its own fluorescence (Cal Orange, in this particular example). Curves 23 correspond to reactions without Reagent 2; Curves 24 corresponds to reactions with 25 nM Reagent 2.

### Example 5—Sites of Methylation/Demethylation Selectively Amplified Using LEL-PCR and Visualized Using Fluorescent Probes

Selective amplification of targets based on the methylation status of a specific site allows for the determination of the methylation status of sites internal to the amplified region. Such internal methylation differences are converted to sequence differences after bisulfite treatment. To model this situation, the experiment depicted FIG. 14 and in Example 3 was repeated using a pair of matched unmethylated targets, one simulating an internal un-methylated CpG after bisulfite conversion within the amplified region ([SEQ ID NO: 11], used in Example 3) and the other simulating the sequence after bisulfite conversion corresponding to the same site if it were methylated. These internal sequence differences were visualized using a fluorescent single mismatched tolerant fluorescent probe.

The sequence of the targets used was as follows:

Matched Simulated Unmethylated Target with an Internal Unmethylated Site:

[SEQ ID NO: 11]
5′CTAAAGCCACCTCCTTACTTTGCCTCCTTCTGCA [symbol] GGTATTCTTTCTC
TTCCGCATGCACCAGTTTGGCCTGCCCAAAATCTGTGATCTTGACATGCT
GCGGTGTTTTCACCAGTACGTTCCTGG3′

Matched Simulated Unmethylated Target with an Internal Methylated Site:

[SEQ ID NO: 15]
5′CTAAAGCCACCTCCTTACTTTGCCTCCTTCTGCA [symbol] GGTATTCTTTCTC
TTCCGCATGCACCAGTTTGGCCTGCCCAAAATCTGTGATCTTGACATGCT
GCGGTGTTTTCACCAGTACGTTCCTGG3′

The simulated unmethylated site within the matched unmethylated target is indicated below in bold and italics. The simulated unmethylated site used for selective amplification is shown underlined and in bold.

### Methylation Mismatch-Tolerant Fluorescent Probe:

[SEQ ID NO: 16]
Quasar 670 5′ CCAAACTGGTGCGGG 3′ BHQ 2

The underline nucleotide in bold is matched to the internal methylated site and mismatched to the internal un-methylated site. The predicted Tm of the probe-amplicon hybrids for the unmethylated and methylated internal site calculated using Visual OMP (DNA Software, Ann Arbor, Mich.) were 47° C. and 57° C.

Amplifications were carried out in replicate reactions in a Stratagene MX3005P thermal cycler (Agilent, Santa Clara, Calif.). PCR assays were performed in a 15 μl volume consisting of 1× Platinum Taq buffer (Invitrogen, Carlsbad, Calif.), 3 mM MgCl2, 250 μM of each deoxynucleotide triphosphate, 50 nM of LATE-PCR SuperSelective primer, 1000 nM for LATE-PCR reverse primer, 0.24×SYBR Green (Invitrogen, Carlsbad, Calif.), 1.5 units of Platinum Taq DNA polymerase (Invitrogen, Carlsbad, Calif.), 25 nM Reagent 2 and 10,000 copies of either matched un-methylated target with an internal methylated site or matched un-methylated target with an internal unmethylated site. The reactions were first incubated at 95° C. for three minutes, followed by 10 cycles of denaturation at 95° C. for 15 seconds and primer annealing at 70° C. for 15 seconds, and primer extension at 80° C. for 30 seconds (note: primer extension was done at 80° C. instead of the customary 72° C.-75° C. to prevent further hybridization events of the SuperSelective primer during extension), one cycle of denaturation at 95° C. for 15 seconds and primer annealing at 60° C. for 15 seconds, and primer extension at 72° C. for 30 seconds, 50 cycles of 95° C. for 15 seconds and primer annealing/extension at 75° C. for 30 seconds, and a melting from 25 C to 95° C. with fluorescent acquisition in the Cal Orange channel (to monitor the fluorescence of Reagent 2) and in the Quasar 670 channel to monitor probe fluorescence. SYBR Green fluorescent intensity was measured during each extension step throughout the course of each reaction to follow the amplification reaction in real time

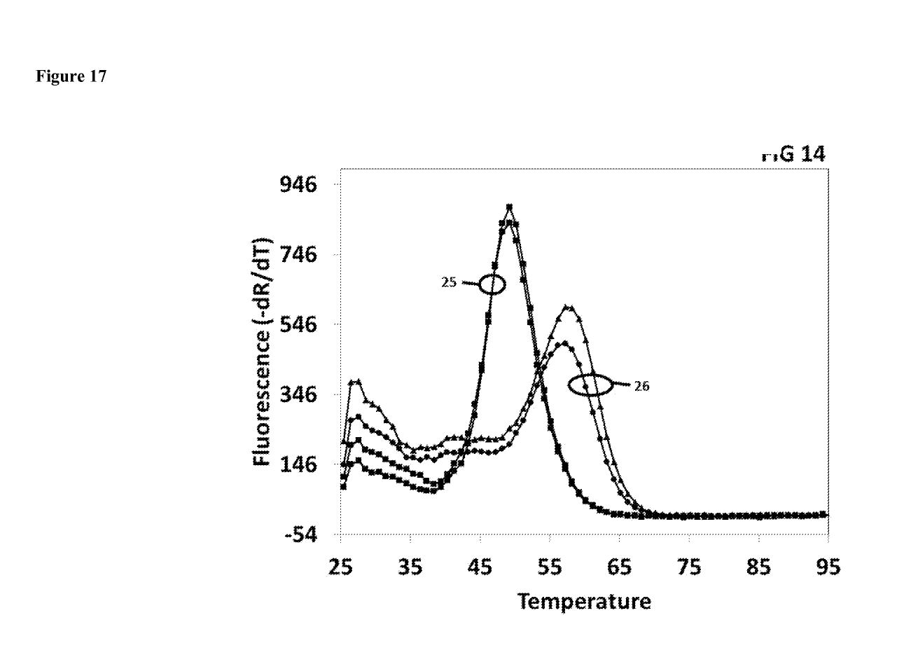

FIG. 17 shows that the probe readily distinguished selectively amplified amplicons containing a simulated internal unmethylated site from those containing the same simulated site but methylated. Curves 25 correspond to amplicons with an internal unmethylated site, Curves 26 correspond to amplicons with an internal methylated site.

### Example 6—Temperature Dependent Reagents to Control the Activity of the Polymerase within a Range of Temperatures

Examples 2 and 4 above illustrate the use of Temperature-Dependent Reagents EP003 and Reagent 2 to control the specificity of Taq DNA polymerase within a range of temperatures where these reagents remain double-stranded simply by changing the concentration of the reagent. This example demonstrates the use of another type of Temperature Dependent Reagent, Hairpin Reagent 1, a double-dabcyl hairpin oligonucleotide, to control the activity of the Taq DNA polymerase. Hairpin Reagent 1 (described in U.S. Pat. No. 7,517,977, included by reference herein in its entirety) comprising a hairpin oligonucleotide having a stem duplex greater than six nucleotides in length and a stabilized stem terminus by 5′ and 3′ terminal dabcyl modifications.

### Hairpin Reagent 1:

[SEQ ID NO: 17]
5′ [5-DABCYL] GAATAATATAG - loop sequence -
CTATATTATTC [DABCYL Q] 3′

The melting temperature of the stem duplex of Hairpin Reagent 1 is 53° C.

The mismatched and matched targets used in Example 3 [SEQ ID NO: 11 and SEQ ID NO: 12] were amplified with Forward EGFR anchor primer [SEQ ID NO: 5—Example 1] and Reverse EGFR primer [SEQ ID NO: 2—Example 1] in the presence of recombinant Taq DNA polymerase supplemented with either Taq antibody or with 1 μM Hairpin Reagent 1 to test whether Hairpin Reagent 1 prevents amplification of these targets during temperature cycling despite these targets being at a starting copy number of 1,000,000 each.

Amplifications were carried out in replicate reactions in a Stratagene MX3005P thermal cycler (Agilent, Santa Clara, Calif.). PCR assays were performed in a 30 μl volume consisting of 1× Platinum Taq buffer (Invitrogen, Carlsbad, Calif.), 3 mM MgCl2, 250 μM of each deoxynucleotide triphosphate, 60 nM Forward EGFR Anchor primer, 60 nM for Reverse EGFR primer, 0.24×SYBR Green (Invitrogen, Carlsbad, Calif.), 1.5 units of Taq DNA polymerase (Invitrogen, Carlsbad, Calif.), 1,000,000 copies of either matched un-methylated target with an internal methylated site or matched un-methylated target with an internal unmethylated site and either 1.5 units of Taq DNA antibody (Invitrogen, Carlsbad, Calif.) or 1 μM Hairpin Reagent 1 (Biosearch, Petaluma, Calif.). The reactions were first incubated at 95° C. for three minutes, followed by 50 cycles of denaturation at 95° C. for 15 seconds and primer annealing at 60° C. for 15 seconds, and primer extension at 72° C. for 30 seconds. SYBR Green fluorescent intensity was measured during each extension step throughout the course of each reaction to follow the amplification reaction in real time.

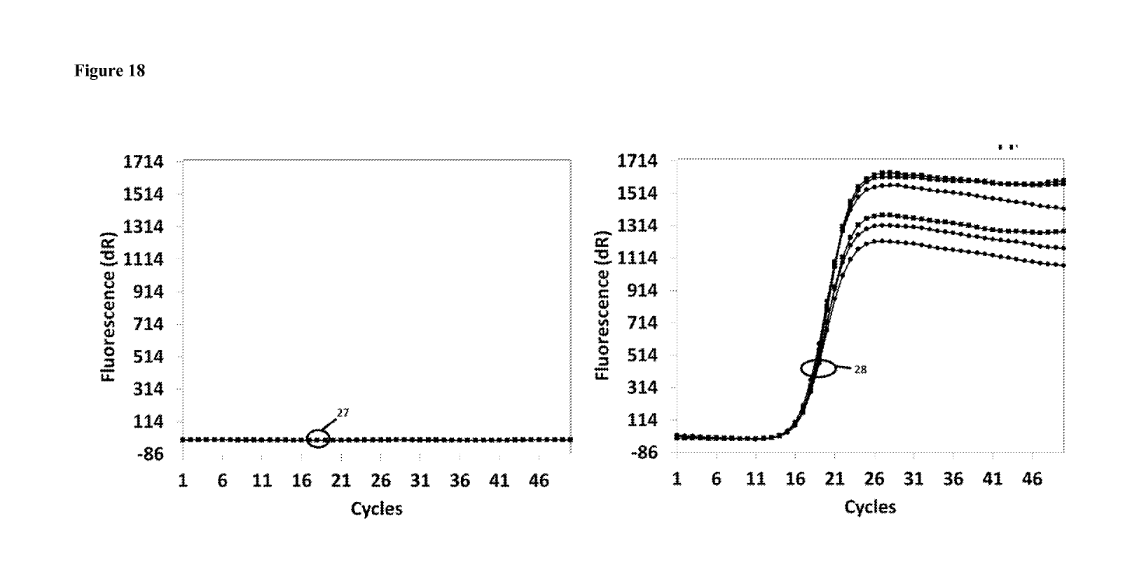

FIG. 18 shows that Hairpin Reagent 1 (Curve 27) successfully prevented amplification over a range of temperature. Control reactions with Taq DNA polymerase and Taq DNA polymerase antibody demonstrate that failure to amplify was due to Hairpin Reagent 1 controlling the activity of Taq DNA polymerase at the annealing temperature (Curve 28). This experiment demonstrate that Temperature-Dependent Reagents rather than precise temperature control can be used to define the activity of Taq DNA polymerase during PCR amplification.

### Example 7—Temperature Imprecise PCR (TI-PCR)

A TI-PCR reaction is carried out using LEL-PCR amplification reaction and the SuperSelective limiting primer and the 5′-extended excess primer and the temperature and cycling conditions described in Example 3. The reaction mixture is optimized by combining 300-1000 nM of a double-dabcylated hairpin oligonucleotide with either 12.5-200 nM of a three dabcyl double-stranded oligonucleotide, such as EP003 (Example 2) or 12.5-200 nM of a double-stranded oligonucleotide labeled with a fluorophore and a quencher, as in Example 4. The double-hairpin oligonucleotide serves as a “hot start-like” inhibitor of polymerase activity prior to amplification and at any time during amplification when the temperature of the reaction vessel falls into Zone 3 of FIG. 19, i.e. approximately 2° C. or more below the temperature needed for hybridization and extension of the Excess primer at its initial low-Tm. The concentration and melting temperatures of the three dabcl double-stranded oligonucleotide or the double-stranded fluorophore/quencher oligonucleotide are optimized to increase the specificity of the DNA polymerase in Zone 2 of FIG. 19. As the two strands of these double-stranded oligonucleotides begin to hybridize at the highest temperatures of Zone 2, the effective double-stranded concentrations of these reactions increase as the temperature of Zone 2 decreases. For this reason, a moderate-Tm primer or a high Tm primer, such as a SuperSelective primer before and after incorporation, can rapidly bind to and extend on its target as the temperature of the reaction falls into Zone 2. In contrast, a low-Tm primer, such as the Excess primer prior to incorporation, requires a lower temperature and longer time to bind to and extend on its template strand. As illustrated in FIG. 19, this is accomplished by delaying the heat pulse for one cycle. However, in contrast to Example 3, the length of this tow temperature step does not need to be precisely controlled in TI-PCR because the activity of the enzyme is inhibited as long as the reaction is in Zone 3. As depicted in FIG. 19, when heat is pulsed back into the system, the total time spent in the lower range of Zone 2 is sufficiently long to allow for initial hybridization and extension of the excess primer. After this thermal cycle the complementary sequences of the limiting (SuperSelective primer) the 5′-extended excess primer is present in the amplicon strands. For this reason, both primers become high-Tm primers which can hybridize and extend rapidly when the reaction enters Zone 2. Thus, the high frequency heat pulse can now be used to exponentially amplify both strands until the limiting primer runs out. Thereafter, high frequency heat pulses can continue to be used for linear amplification of just the excess primer Strand.

The reaction is paused, or completed by delaying the pulsation of heat at the desired cycle. The temperature of the reaction decreases at a rate that depends on the ambient temperature. As before, the activity of the DNA polymerase is inhibited by the double-dabcylated hairpin oligonucleotide as soon as Zone 3 is reached. One or more low-Tm double labeled fluorescent probes, or quencher only probes that have been present throughout the reaction bind to the accumulated single-strands and generate a characteristic signal. If a double-strand DNA binding dye is also present in the reaction, it too binds the double-stranded amplicon molecules and the single-stranded amplicon molecules having bound probes. These closed-tube methods of amplicon analysis are described U.S. patent application publication numbers: 2012/0282611 and 2013/0095479, each of which is hereby incorporated by reference in its entirety.

### Example 8: LEL-PCR Applied to Detection of Drug Resistant Tuberculosis

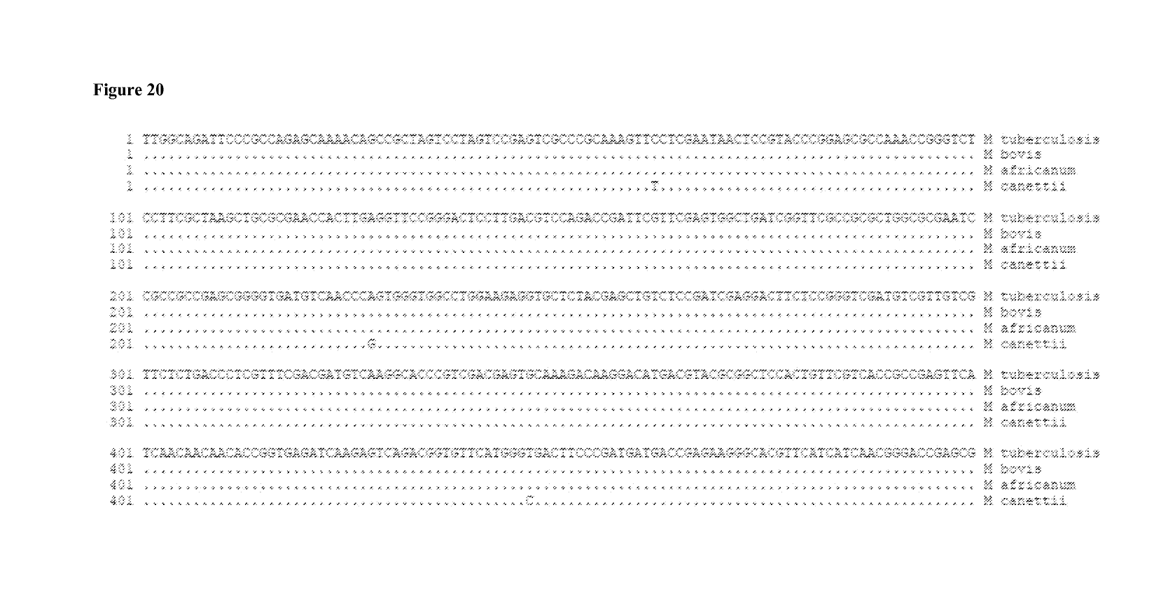

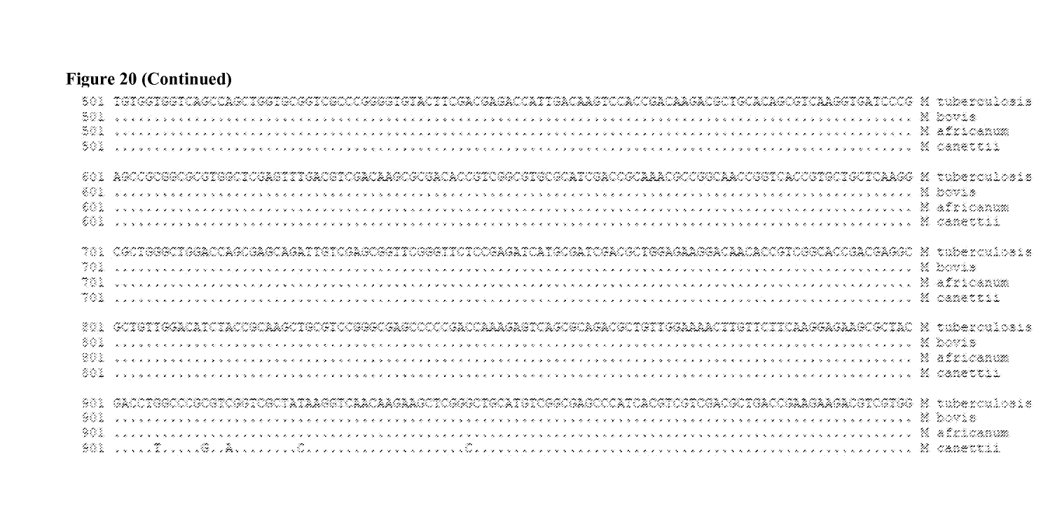

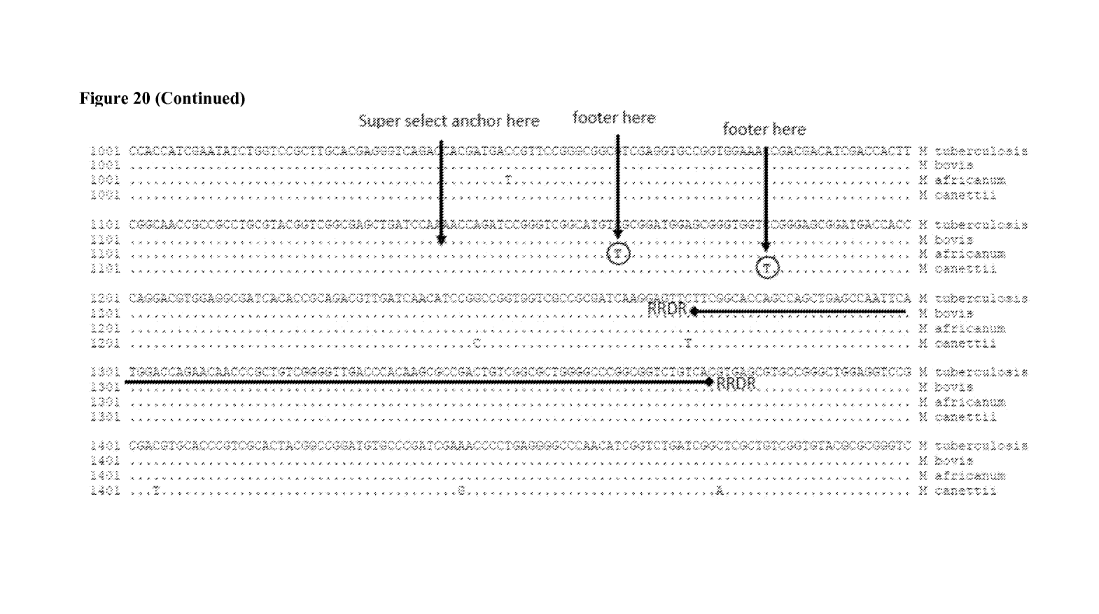

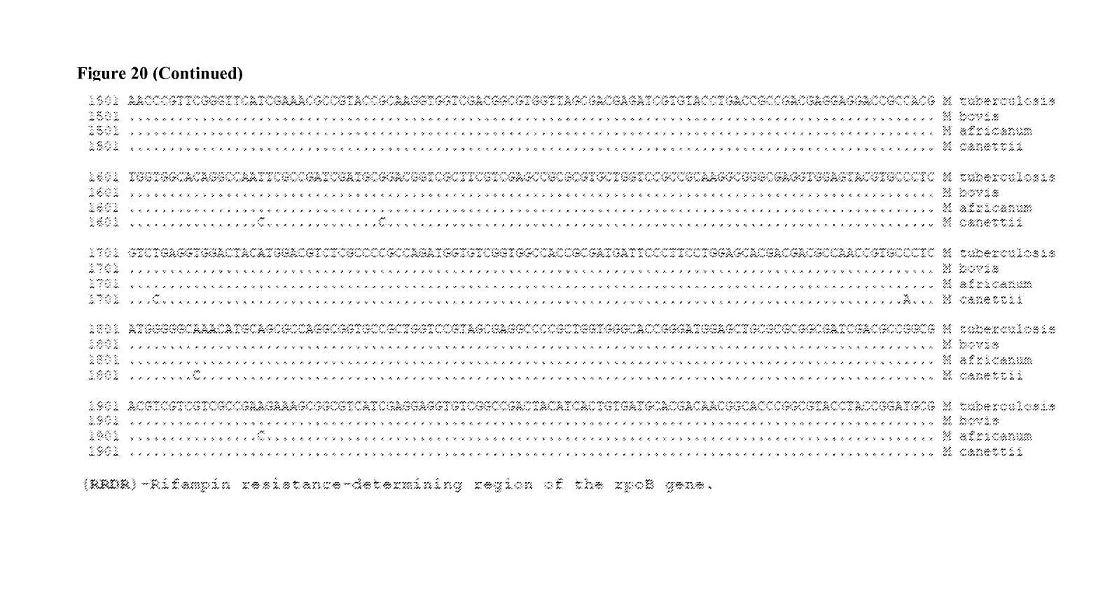

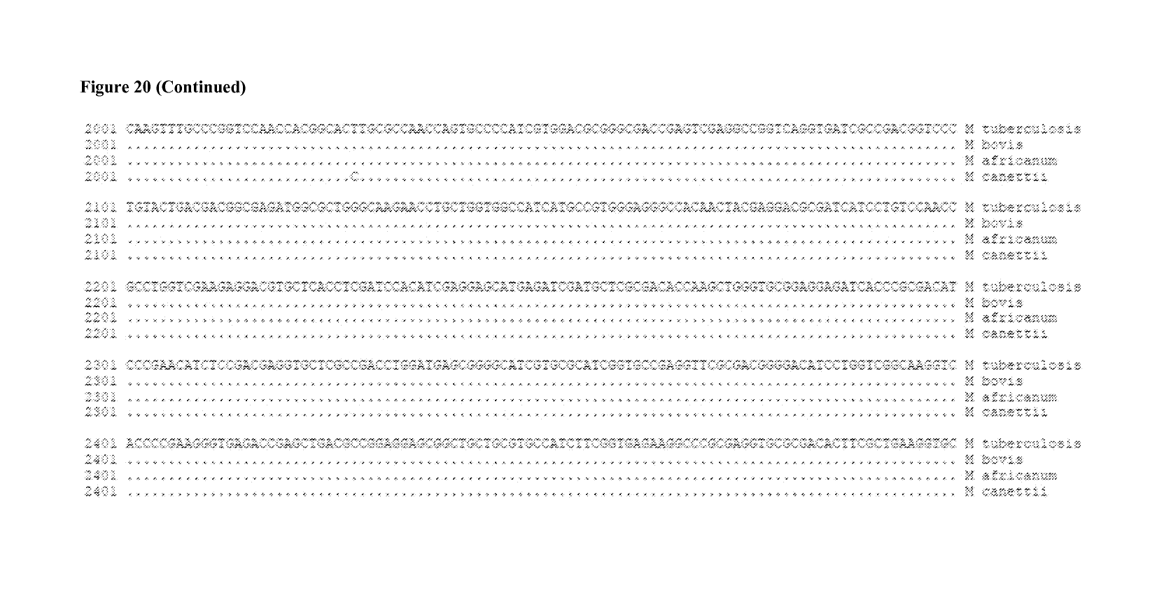

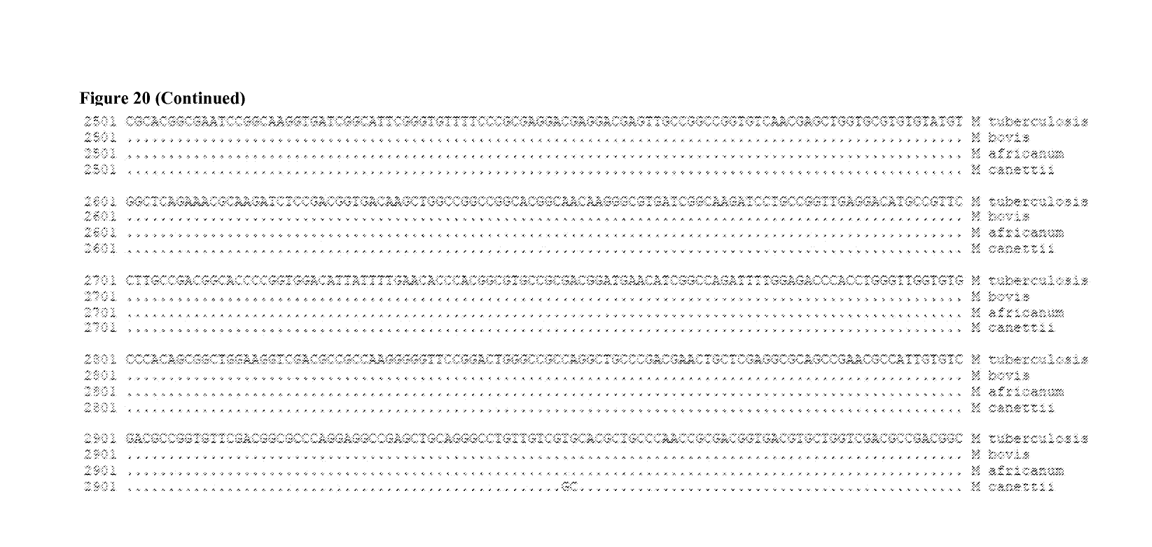

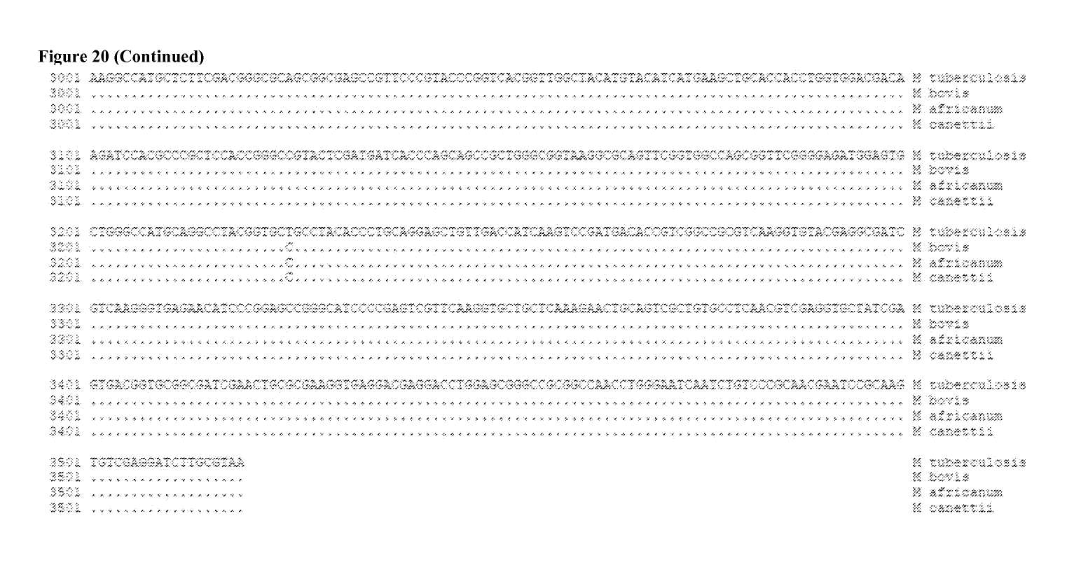

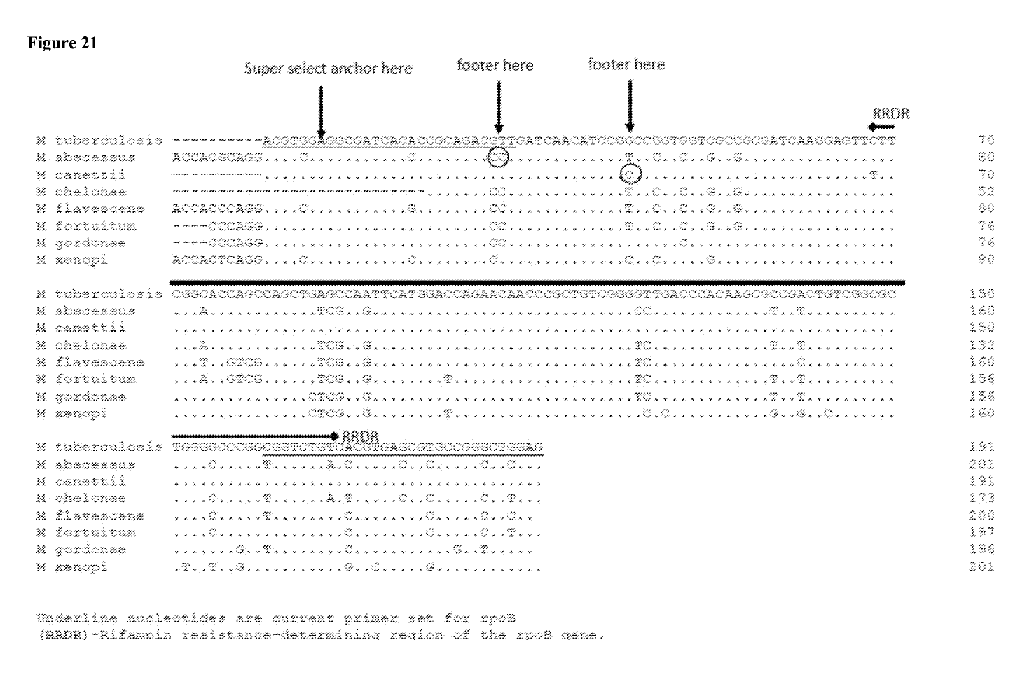

Antibiotic resistance in tuberculosis (among many other pathogens) is due to the presence of mutations in one or more gene targets. The RRDR portion of the rpoB gene of M. tuberculosis is of particular importance because the rpoB gene product is normally sensitive to rifampicin and its family of antibiotics, but many mutations in the RRDR are known to result in resistance to these antibiotics. It is therefore of interest to be able to rapidly, accurately, and inexpensively screen human samples for the presence/absence of M. tuberculosis as well as its drug resistant status. LEL-PCR is a useful technology for detection and diagnosis M. tuberculosis DNA because of its very high sensitivity and specificity, even in the presence of DNA from other organisms, including host (human) DNA. In this case the sequence-specific forward primer, for instance a SuperSelective primer, is positioned over a sequence flanking the RRDR region. The exact target sequence is chosen because it is unique to M. tuberculosis, i.e., it differs in other species of mycobacteria, as well as in the host DNA. The reverse primer also flanks the RRDR of rpoB, but binds to the opposite strand of target. Exemplary primer positions are provided in the alignments presented in FIGS. 20 and 21.

As described above, the initial concentration dependent Tm of the anchor of the forward primer is greater than 5 degrees higher or greater than 10 degrees higher than the initial concentration-dependent Tm of the reverse primer. The initial concentration of the reverse primer is at least 2-fold or at least 5 fold greater than that of forward primer. The reaction is begun with one or more rounds of linear synthesis of the single-strand generated by extension of just the forward primer. The resulting strand then becomes the template for a single round of synthesis achieved by binding the reverse primer at a much lower annealing temperature. The strands resulting from these two steps contain the complements of the full length forward primer and the full length reverse primer, allowing subsequent rounds of exponential amplification to be carried out at temperature that is too high for subsequent binding of the initial binding sequences of both the forward and the reverse primers. Because the initial concentration of the forward primer is lower than that of the reverse primer, the forward primer is used up before the reverse primer and reaction thereafter carries out linear amplification of only the reverse primer strand. Sequences within the RRDR single-stranded amplicon are identified by hybridization of appropriate probes at a temperature below the melting temperature of the full length forward primer to its template strand during the exponential phase of the reaction.

### Example 9: Use of LEL-PCR for Selective Amplification of the Cytochrome C Oxidase Subunit I Gene in Mitochondrial DNA

In their recent paper entitled “Are “universal” DNA, primers really universal?” (Journal of Applied Genetics, DOI 10.1007/s13353-014-0218-9) Pranay Sharma and Tsuyoshi Kobayashi state the following “The mitochondrial cytochrome c oxidase subunit I (COI) gene has been accepted as the standard taxon barcode for most animal groups due to its robustness, reliability and sufficient resolution to identify a range of organisms. The role of a phylogenetic study is to aid research in locating where species fit in a unified tree of life. To achieve this, one has to amplify the gene of interest using primers. The universal primers designed by Folmer et al., Mol. Mar. Biol. Biotechnol. 3:294-299 (1994), LCO 1490 and FICO 2198, with 25 and 26 base pairs (bp) in length, respectively, are widely used primers in the animal kingdom. A Folmer primer amplifies the first half of the COI gene, which is a gene fragment of length approximately 700 bp. The success rate of the primers in amplifying the COI fragment in highly divergent animal species has been remarkable due to its conserved 3′ ends.”

In the present example we use the forward primer LCO 1490 and the reverse primer HCO 2198 described in Folmer et al., Mol. Mar. Biol. Biotechnol. 3:294-299 (1994) (incorporated by reference in its entirety) as the starting point for designing sets of SuperSelective limiting primers and excess primers-with-5′ unhybridized-tails to serve as the primer pairs in sets of LEL-PCR reactions that are designed to distinguish genera within a taxonomic family. In other words, one pair of primers within our sets of primers will be best matched to all species within the same Genus, while another pair of primers within our sets of primers will be best matched to another Genus within the same Family. It is assumed that a trained naturalist or zoologist will be able to distinguish Families of organisms with in an Order. Viewed in this way, one pair of primers will serve to amplify all species within a particular Genus. The individual species within that Genus will be distinguished by their individual “fluorescent signatures” generated by end-point hybridization of a universal set of “Lights-On/Lights-Off” probes in one or more colors (as described in “Virtual Barcoding using LATE-PCR and Lights-On/Lights-Off Probes: Identification of Nematode Species in a Closed-Tube Reaction” by_Lisa M. Rice, Arthur H. Reis, Jr., and Lawrence J. Wangh, in Mitochondrial DNA, in press as of July, 2014).

Sets of primer pairs for LEL-PCR amplification of any species within a particular Genus can be arranged in a two dimensional array, such as a 96-well, or 384-well PCR plate such that each well contains more than one pair of primers. Low temperature probes, such as Lights-On/Lights-Off probes in the same color can be included in the reaction mixture and can be designed to hybridize to a coded sequence within either the bridge portion or the 5′-tail portion of the SuperSelective limiting primer, or the 5′-tailed excess primer, or both, when the temperature of the reaction is dropped at end-point. The fluorescent signature generated by melting these probes off will be indicative of the coded sequence present in the primers and will thereby indicate the exact primer sequences which served to amplify the species of that Genus.

Strategies very similar to that described above can also be designed for Genus and Species identification of bacteria. For instance the 5′-tailed excess primer can hybridize to one of the conserved sequences in the 16s ribosome RNA gene target and the SuperSelective primer can have its anchor located to the another conserved sequence of the 16s ribosomal RNA gene target while the foot of the primer hybridizes to an adjacent genus specific sequence. A particular pair of primers designed in this way will identify at the genus and species level which bacterium is most prevalent within a population. However, other pairs of primers within such a set of primers will identify other bacterial Genera/Species present in a mixed population.

### Example 10: LEL-PCR Using Tailed Primers

Two monoplex LEL-PCR amplification reactions (“LEL-PCR 1” and “LEL-PCR 2”) were performed in which both the LEL-PCR limiting primer and the LEL-PCR excess primer included 5′ tail sequences that were not complementary to the target at the start of amplification, but that became complementary to the amplified product as a result of amplification. Such non-complementary sequences are be referred to herein as “primer 5′ tail sequences.”

Monoplex LEL-PCR amplification reactions were carried out in 1× Invitrogen PCR buffer (Life Technologies, Grand Island, N.Y.), 3 mM MgCl₂, 250 nM dNTPs, 0.24×SYBR-Green, 800 nM of a Temperature Dependent Reagent (SEQ ID NO: 18), 2 units Taq DNA polymerase (Life Technologies, Grand Island, N.Y.), 50 nM LEL-PCR limiting primer, 1 μM LEL-PCR excess primer, 500 nM hybridization probe and 10,000 copies of DNA target. Amplification reactions were carried out in a Stratagene MX3000P thermocycler (Agilent Technologies, Santa Clara, Calif.). Thermocycling conditions were 3 minutes at 95° C. for 1 cycle; 10 seconds at 95° C./30 seconds at 72° C. for 10 cycles; 20 seconds at 60° C. for 1 cycle; 10 seconds at 95° C./54 seconds at 78° C. for 50 cycles, with fluorescence acquisition at 78° C. At the end of amplification, the temperature was lowered 1° C. every 30 seconds from 60° C. to 25° C. for probe hybridization. Probes were then melted off the template by raising the temperature 1° C. every 30 seconds from 25° C. to 95° C., with fluorescent acquisition at every temperature step.

### Temperature Dependent Reagent

[SEQ ID NO: 18]
5′ BHQ2 GAATAATATAGCCCCCCCCCCCCCCCCCCCCCCCCCCCCCTA
TATTATTC Biosearch Blue 3′

The underlined primer sequences correspond to the 5′ region of the primer that is not complementary to the target at the start of the reaction. Concentration-adjusted primer melting temperatures were calculated using Visual OMP software, version 7.8.42 (DNA Software, Ann Arbor, Mich.).

### LEL-PCR 1 Limiting Primer

[SEQ ID No: 19]
5′ TGGCCATGGCAATCAGTTGCTGTTACCTGTCAAAAGGATACTACACC
TC 3′

In silico concentration-adjusted melting temperature of 50 nM LEL-PCR 1 limiting primer hybridized to the target at the start of the reaction (Tm_{L}⁰) was 73.3° C.

In silico concentration-adjusted melting temperature of 50 nM LEL-PCR 1 limiting primer hybridized to the amplicon (Tm_{L}^{r}) was 79.6° C.

### LEL-PCR 1 Excess Primer

[SEQ ID NO: 20]
5′ AATCTCCTCCTCCTCCTTACCTATAAAAATTTTCGGCCAAGGGGATA
T 3′

In silico concentration-adjusted melting temperature of 1 μM LEL-PCR 1 excess primer hybridized to the target at the start of the reaction (Tmₓ⁰) was 56.4° C.

In silico concentration-adjusted melting temperature of 1 μM LEL-PCR 1 excess primer hybridized to the amplicon (Tmₓ^{r}) was 79.6° C.

The melting temperature of the LEL-PCR 1 amplicon (Tm^{A}) was 86.8° C.

LEL-PCR 1 primers are distinct from LATE-PCR primers at least because they do not meet the LATE-PCR design criteria that specifies TmA-Tmₓ⁰<25° C. For the LEL-PCR 1 primers, TmA-Tmₓ⁰=30.4° C.

### LEL-PCR 1 Hybridization Probe

[SEQ ID NO: 21]
5′ BHQ2 ATCCATATGATAAATTAT-3′ CalRed610

### LEL-PCR 2 Limiting Primer

[SEQ ID NO: 22]
5′ TGGCCAGTCACAGCTATAACATGTCAACGGGAACAGCCACCAA 3′

In silico concentration-adjusted melting temperature of 50 nM LEL-PCR 2 limiting primer hybridized to the target at the start of the reaction (Tm_{L}⁰) was 73.5° C. In silico concentration-adjusted melting temperature of 50 nM LEL-PCR 2 limiting primer hybridized to the amplicon (Tm_{L}^{r}) was 79.3° C.

### LEL-PCR 2 Excess Primer

[SEQ ID NO: 23]
5′ AATCCTCCTCCTCCTTAAAAACTTACGGCCCAGTGGAAATTGATC 3′

In silico concentration-adjusted melting temperature of 1 μM LEL-PCR 2 excess primer hybridized to the target at the start of the reaction (Tmₓ⁰) was 60.2° C.

In silico concentration-adjusted melting temperature of 1 μM LEL-PCR 2 excess primer hybridized to the amplicon (Tmₓ^{r}) was 79.3° C.).

Melting temperature of the LEL-PCR 2 amplicon (Tm^{A}) was 87.3° C.

LEL-PCR 2 primers are distinct from LATE-PCR primers at least because they do not meet the LATE-PCR design criteria that specifies TmA-Tmₓ⁰<25° C. For LEL-PCR 2 primers, TmA-Tmₓ⁰=27.1° C.

### LEL-PCR 2 Hybridization Probe

[SEQ ID NO: 24]
5′ BHQ1 CAGGACAGTTTTT 3′ Cal-Orange 560

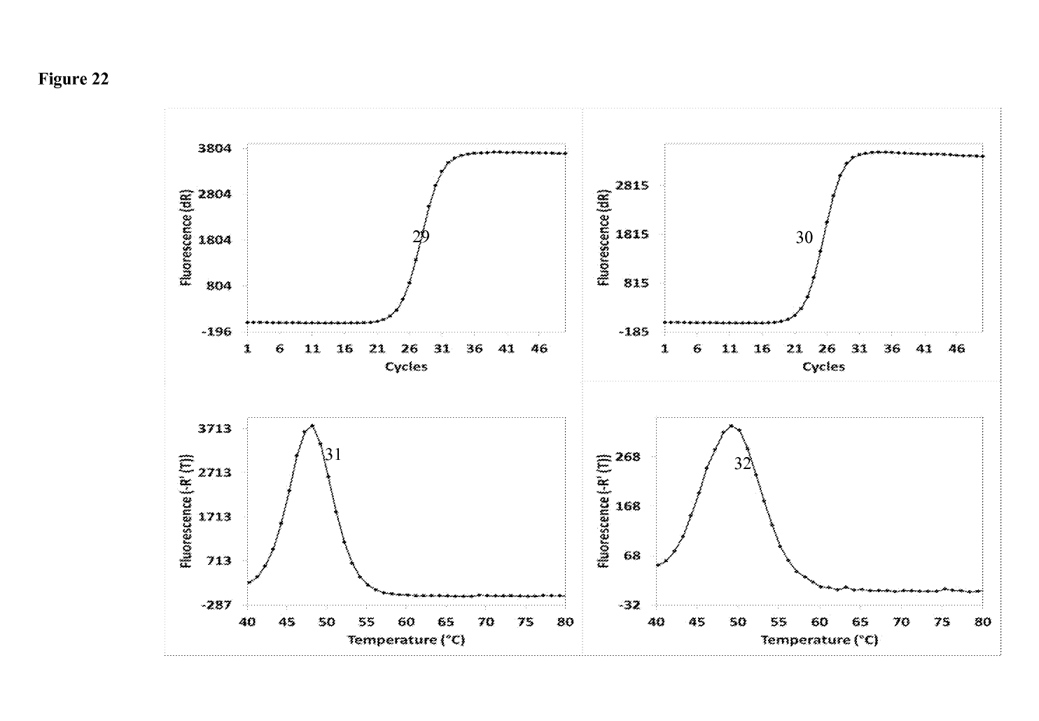

As seen in FIG. 22, when LEL-PCR was performed using such primers, LEL-PCR double-stranded products were detected in real-time using SYBR Green and single-stranded products were detected at endpoint using melting curve analysis of probe hybridization signals. The curves shown in FIG. 22 are as follows: curve 29, LEL-PCR 1 SYBR Green amplification; curve 30, LEL-PCR 2 SYBR Green amplification; curve 31, LEL-PCR 1 probe hybridization signal, Cal Red 610 channel; curve 32, LEL-PCR 2 probe hybridization signal, Cal Orange 560 channel. Probe hybridization signals are shown as negative first derivatives of fluorescence signals relative to temperature. Each curve corresponds to the average of three replicates samples.

### Example 11: Use of a Temperature Dependent Reagent on Multiplex LEL-PCR

The use of a Temperature Dependent Reagent referred to as “ThermaGo-3” in multiplex LEL-PCR amplification reactions was examined. ThermaGo-3 is a modified double-stranded DNA oligonucleotide construct that improves amplification specificity and amplicon yield in PCR amplifications (U.S. Provisional Patent application No. 62/136,048, which is hereby incorporated by reference in its entirety, and SEQ ID NOs: 25 and 26).

No target control LEL-PCR amplification reactions were carried out in 1× Invitrogen PCR buffer (Life Technologies, Grand Island, N.Y.), 3 mM MgCl₂, 250 nM dNTPs, 0.24×SYBR-Green, 800 nM Temperature Dependent Reagent (SEQ ID 17), 2 units Taq DNA polymerase (Life Technologies, Grand Island, N.Y.), 50 nM LEL-PCR 2 limiting primer, 1 μM LEL-PCR 2 excess primer, 500 nM LEL-PCR 2 Cal-Orange 560 hybridization probe in the absence or presence of 100 nM of each oligonucleotide of ThermaGo-3. Amplification reactions were carried out in a Stratagene MX3000P thermocycler (Agilent Technologies, Santa Clara, Calif.). Thermocycling conditions were 3 minutes at 95° C. for 1 cycle; 10 seconds at 95° C./30 seconds at 72° C. for 10 cycles; 20 seconds at 60° C. for 1 cycle; 10 seconds at 95° C./54 seconds at 78° C. for 50 cycles, with fluorescence acquisition at 78° C. At the end of amplification, the temperature was lowered 1° C. every 30 seconds from 60° C. to 25° C. for probe hybridization. Probes were then melted off the template by raising the temperature 1° C. every 30 seconds from 25° C. to 95° C., with fluorescent acquisition at every temperature step.

### ThermaGo-3

Upper Strand:
[SEQ ID NO: 25]
5′ GAGCAGACTCGCACTGAGGTA 3′ Biosearch Blue
Lower Strand:
[SEQ ID NO: 26]
5′ BHQ-2 TACCTCAGTGCGAGTCTGCTC 3′ Biosearch Blue

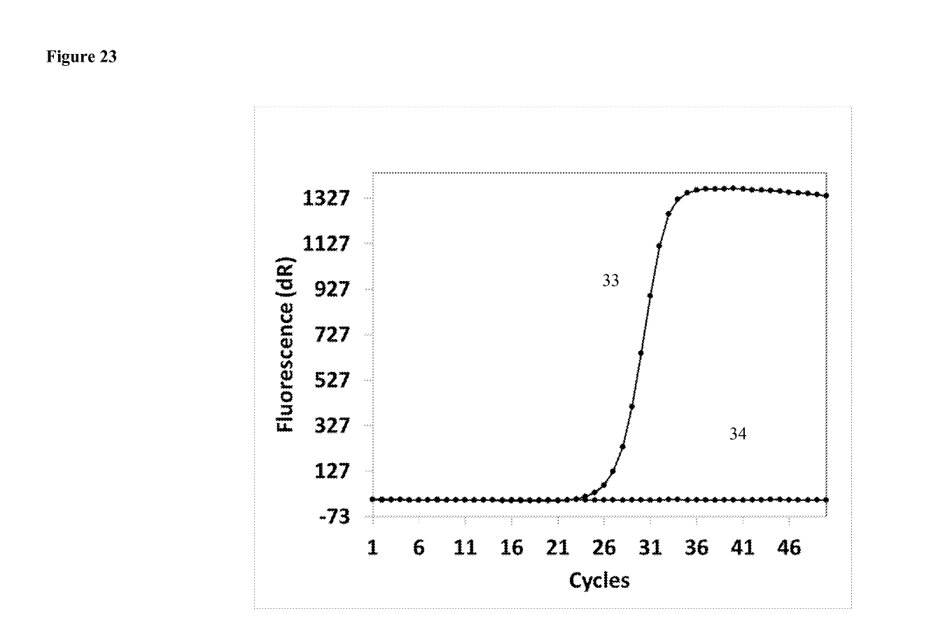

As seen in FIG. 23, addition of 100 nM ThermaGo-3 inhibited formation of primer dimers generated in no target control LEL-PCR amplifications. Double-stranded LEL-PCR primer dimers in no target control assays were detected in real-time using SYBR Green. Amplification products from such no target control assays were determined to be primer dimers and not the result of target contamination based on the melting temperature of the resulting amplicon products and the absence of probe hybridization signals corresponding to the amplification product generated in the presence of target. The curves shown in FIG. 23 are as follows: curve 33, SYBR Green amplification in the absence of ThermaGo-3; curve 34, SYBR Green amplification in the presence of 100 nM ThermaGo-3. Probe hybridization signals are shown as negative first derivatives of fluorescence signals relative to temperature. Each curve corresponds to the average of three replicates samples.

Multiplex LEL-PCR reactions that included both LEL-PCR 1 and LEL-PCR 2 amplification was carried out in the absence or presence of 100 nM ThermaGo-3. Multiplex LEL-PCR amplification reactions were carried out in 1× Invitrogen PCR buffer (Life Technologies, Grand Island, N.Y.), 3 mM MgCl₂, 250 nM dNTPs, 0.24×SYBR-Green, 800 nM Temperature Dependent Reagent (SEQ ID No: 17), 2 units Taq DNA polymerase (Life Technologies, Grand Island, N.Y.), 50 nM LEL-PCR 1 limiting primer, 50 nM LEL-PCR 2 limiting primer, 1 μM LEL-PCR 1 excess primer, 1 μM LEL-PCR 2 excess primer, 500 nM LEL-PCR 1 Cal-Red 610 hybridization probe, 500 nM LEL-PCR 2 Cal-Orange 560 hybridization probe, and 10,000 copies of DNA target for each primer set in the absence or presence of 100 nM of each strand of ThermaGo-3. Amplification reactions were carried out in a Stratagene MX3000P thermocycler (Agilent Technologies, Santa Clara, Calif.). Thermocycling conditions were 3 minutes at 95° C. for 1 cycle; 10 seconds at 95° C./30 seconds at 72° C. for 10 cycles; 20 seconds at 60° C. for 1 cycle; 10 seconds at 95° C./54 seconds at 78° C. for 50 cycles, with fluorescence acquisition at 78° C. At the end of amplification, the temperature was lowered 1° C. every 30 seconds from 60° C. to 25° C. for probe hybridization. Probes were then melted off the template by raising the temperature 1° C. every 30 seconds from 25° C. to 95° C., with fluorescent acquisition at every temperature step.

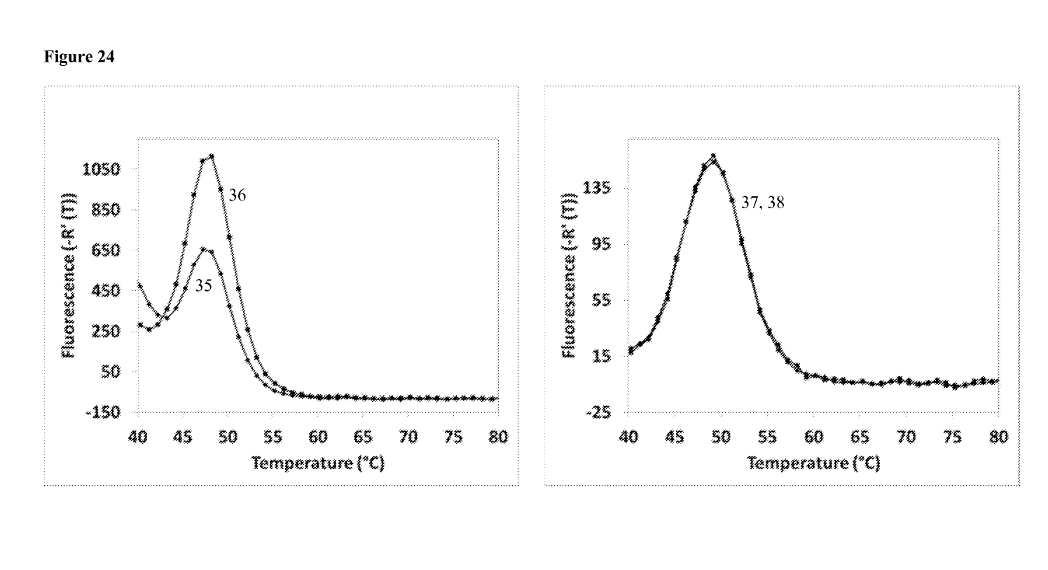

As seen in FIG. 24, single-stranded LEL-PCR multiplex amplification products were detected at end-point point using melting curve analysis of probe hybridization signals. The curves in FIG. 24 are as follows: curve 35, LEL-PCR 1 probe hybridization signal without ThermaGo-3, Cal Red 610 channel; curve 36, LEL-PCR 1 probe hybridization signal in the presence of 100 nM ThermaGo-3, Cal Red 610 channel; curve 37, LEL-PCR 2 probe hybridization signal without ThermaGo-3, Cal Orange 560 channel; curve 38, LEL-PCR 2 probe hybridization signal in the presence of 100 nM ThermaGo-3, Cal Orange 560 channel. Probe hybridization signals are shown as the negative first derivatives of fluorescence signals relative to temperature. Each curve corresponds to the average of three replicates samples.

### Example 12: Use of Oligonucleotides Complementary to the 3′ End of LEL-PCR Limiting Primers

In some embodiments, during LEL-PCR the limiting primer (Tm_{L}⁰=70° C.-72° C.) first hybridizes and extends on the target sequence at an annealing/extension temperature of 72° C. for one to ten cycles in the absence of LEL-PCR excess primer extension (Tmₓ⁰=60° C.) to generate limiting primer strands. The reaction temperature is then lowered to an annealing/extension temperature of 60° C. for one cycle to allow hybridization and extension of the LEL-PCR excess primer on the limiting primer Strands. The annealing/extension temperature is then raised to 78° C.-80° C. for 40-60 cycles to carry out the exponential portion of LEL-PCR amplification. In some cases, the 60° C. annealing/extension temperature may not be sufficiently stringent to prevent mis-priming of the LEL-PCR limiting primer (Tm_{L}⁰=70° C.-72° C.), which may result in some non-specific product formation. Formation of such non-specific products can be inhibited by addition of a Temperature Dependent Reagent (e.g., ThermaGo-3, Example 11). Another strategy to inhibit formation of non-specific products is to use of complementary oligonucleotides that bind to the 3′ end of the LEL-PCR limiting primer during the transition from the 72° C. to the 60° C. annealing/extension temperature. Hybridization of such an oligonucleotide to the 3′ end of the LEL-PCR limiting primer during this step forms a blunt-ended double stranded hybrid that inhibits the binding of the LEL-PCR limiting primer to other targets in the reaction.

In a proof-of-principle experiment, a limiting primer blocking oligonucleotide to the LEL-PCR 1 limiting primer was designed to have a Tm of ˜63° C. which is low enough to not interfere with extension of the LEL-PCR excess primers at the 72° C. and 78° C. annealing/extension temperatures.

Monoplex LEL-PCR amplification reactions were carried out in 1× Invitrogen PCR buffer (Life Technologies, Grand Island, N.Y.), 3 mM MgCl₂, 250 nM dNTPs, 0.24×SYBR-Green, 800 nM Temperature Dependent Reagent (SEQ ID 17), 2 units Taq DNA polymerase (Life Technologies, Grand Island, N.Y.), 50 nM corresponding LEL-PCR limiting primer, 1 μM corresponding LEL-PCR excess primer, 500 nM corresponding hybridization probe and 10,000 copies the DNA target in the presence or absence of 100 nM limiting primer blocking oligonucleotide 1 [SEQ ID NO: 27]. Amplification reactions were carried out in a Stratagene MX3000P thermocycler (Agilent Technologies, Santa Clara, Calif.). Thermocycling conditions were 3 minutes at 95° C. for 1 cycle; 10 seconds at 95° C./30 seconds at 72° C. for 10 cycles; 20 seconds at 60° C. for 1 cycle; 10 seconds at 95° C./54 seconds at 78° C. for 50 cycles, with fluorescence acquisition at 78° C. At the end of amplification, the temperature was lowered 1° C. every 30 seconds from 60° C. to 25° C. for probe hybridization. Probes were then melted off the template by raising the temperature 1° C. every 30 seconds from 25° C. to 95° C., with fluorescent acquisition at every temperature step.

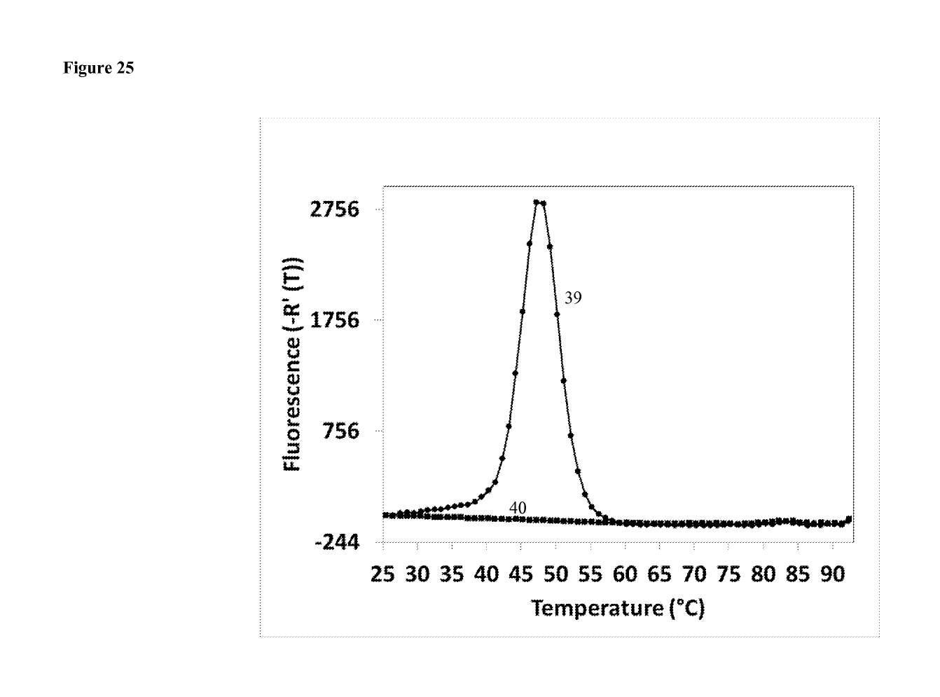

As seen in FIG. 25, addition of 100 nM limiting primer blocking oligonucleotides to a monoplex LEL-PCR reaction consisting of LEL-PCR 1 primers and probe prevented amplification of LEL-PCR products. This result demonstrates the efficacy of these oligonucleotides to inhibit mis-priming by LEL-PCR limiting primers. The curves in FIG. 25 are as follows: curve 39, LEL-PCR 1 probe hybridization signal in the absence of 100 nM limiting primer blocking oligonucleotides, Cal Red 610 channel; curve 40, LEL-PCR 1 probe hybridization signal in the presence of 100 nM limiting primer blocking oligonucleotides, Cal Red 610 channel. Probe hybridization signals are shown as the negative first derivatives of fluorescence signals relative to temperature. Each curve corresponds to the average of three replicates samples.

Limiting Primer Blocking Oligonucleotide 1 Sequence (3′ End Blocked with a C-3 Carbon Spacer)

[SEQ ID NO: 27]
5′ GAGGTGTAGTATCCTTTTGACAGGTAA-C3 3′

In silico concentration-adjusted melting temperature of 100 nM μM limiting primer blocking oligonucleotides hybridized to 50 nM LEL-PCR 1 limiting primer (Tmₓ⁰) was 62.5° C.

### Example 13: LEL-PCR Amplification of a GC Rich Genomic Template

PCR amplification from GC-rich genomes can be challenging due to stable secondary structures and difficulties in making low melting primers. The use of LEL-PCR in conjunction with GC rich templates was therefore tested.

Monoplex LEL-PCR amplification reactions were carried out in 1× Invitrogen PCR buffer (Life Technologies, Grand Island, N.Y.), 3 mM MgCl₂, 300 nM dNTPs, 0.24×SYBR-Green, 600 nM Temperature Dependent Reagent (SEQ ID NO: 17), 1.25 units Taq DNA polymerase (Life Technologies, Grand Island, N.Y.), 50 nM LEL-PCR 3 limiting primer, 1 μM LEL-PCR 3 excess primer, 100 nM Quasar 670 LEL-PCR 3 hybridization probe and 10,000 copies Mycobacterium tuberculosis genomic DNA target. Amplification reactions were carried out in a Stratagene MX3000P thermocycler (Agilent Technologies, Santa Clara, Calif.). Thermocycling conditions were 1 minute at 97° C. for 1 cycle; 7 seconds at 97° C./45 seconds at 69° C. for 10 cycles; 20 seconds at 60° C. for 1 cycle; 7 seconds at 97° C./45 seconds at 78° C. for 50 cycles with fluorescence acquisition at 78° C. At the end of amplification, the temperature was lowered 1° C. every 30 seconds from 70° C.-25° C. for probe hybridization. Probes were then melted off by raising the temperature 1° C. every 33 seconds from 25° C.-100° C., with fluorescent acquisition at every temperature step.

The primer sequences that are underlined below correspond to the 5′ region of the primer that is not complementary to the target at the start of the reaction (“primer 5′ tail sequence”). Concentration-adjusted primer melting temperatures were calculated using Visual OMP software, version 7.8.42 (DNA Software, Ann Arbor, Mich.).

### LEL-PCR 3 Limiting Primer

[SEQ ID NO: 28]
5′ TCGTGAATACCTCCAGCTCGGCACCCTCACGTGACAGACCG 3′

In silico concentration-adjusted melting temperature of 50 nM LEL-PCR 3 limiting primer hybridized to the target (Tm_{L}⁰) was 70.0° C.

In silico concentration-adjusted melting temperature of 50 nM LEL-PCR 3 limiting primer hybridized to the amplicon (Tm_{L}^{r}) was 83.0° C.

### LEL-PCR 3 Excess Primer 3

[SEQ ID NO: 29]
5′ CGAGTCCATCACCTCGCGATCACACCACAGACGTT 3′

In silico concentration-adjusted melting temperature of 1 μM LEL-PCR excess primer 3 hybridized to the target (Tmₓ⁰) was 61.0° C.

In silico concentration-adjusted melting temperature of 1 μM LEL-PCR excess primer 3 hybridized to the amplicon (Tmₓ^{r}) was 81.0° C.

Melting temperature of the LEL-PCR 3 amplicon (Tm^{A}) was 95.6° C.

The LEL-PCR 3 primers can be distinguished from LATE-PCR primers at least because they do not meet the LATE-PCR design criteria that specifies TmA-Tmₓ⁰<25° C. For LEL-PCR 3 primers, TmA-Tmₓ⁰=34.6° C.

### LEL-PCR 3 Hybridization Probe

[SEQ ID NO: 30]
5′ BHQ2-TCAGGTCCATGAATTGGCTCAGA 3′ QSR670

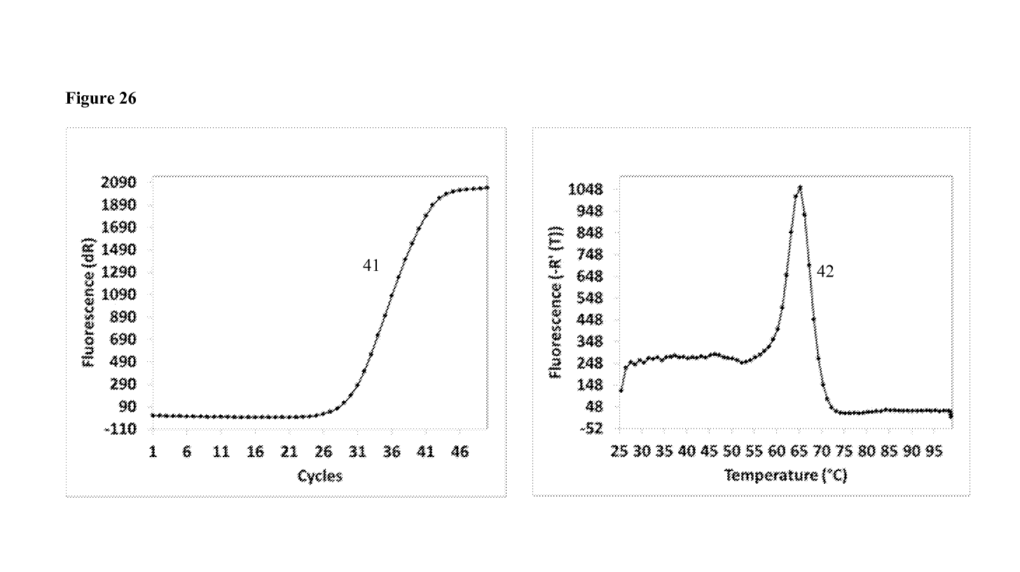

As seen in FIG. 26, monoplex LEL-PCR amplification was successfully performed using tailed LEL-PCR limiting and excess primers to amplify Mycobacterium tuberculosis genomic DNA (approximately 65.6% GC content) as a template. The LEL-PCR 3 primers target a 208 base pair region in the rpoB gene (64% GC). Monoplex LEL-PCR double-stranded products were detected in real-time using SYBR Green and single-stranded products were detected at endpoint using melting curve analysis of probe hybridization signals. The curves shown in FIG. 26 are as follows: curve 41, SYBR Green amplification; curve 42 probe hybridization signal, Quasar 670 channel. Probe hybridization signals are shown as negative first derivatives of fluorescence signals relative to temperature. Each curve corresponds to the average of three replicates samples.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of amplifying a target nucleic acid sequence in a target nucleic acid molecule comprising the steps of:
(a) forming a reaction solution comprising the target nucleic acid molecule, a forward primer, a reverse primer and amplification reagents, wherein:
(i) the forward primer has partial complementarity to a nucleic acid sequence on the 3′ end of the target nucleic acid sequence;
(ii) the reverse primer has partial identity to a nucleic acid sequence on the 5′ end of the target nucleic acid sequence;
(iii) the melting temperature for the reverse primer on the target nucleic acid sequence is lower than the melting temperature for the forward primer on the target nucleic acid sequence; and
(iv) the reverse primer is present in the reaction solution at a higher concentration than the forward primer;
(b) subjecting the reaction solution to one or more linear amplification cycles comprising an annealing temperature that is lower than the melting temperature of the forward primer on the target nucleic acid sequence and higher than the melting temperature of the reverse primer on the target nucleic acid sequence;
(c) subjecting the reaction solution to one or more low annealing temperature amplification cycles comprising an annealing temperature that is lower than the melting temperature of the reverse primer on the target nucleic acid sequence;
(d) subjecting the reaction solution to one or more LATE-PCR amplification cycles comprising an annealing temperature that is above the melting temperatures for the forward primer and the reverse primer on the target nucleic acid sequence and below the melting temperature for the forward primer and the reverse primer on perfectly complementary nucleic acid sequences.

2. The method of claim 1, wherein the forward primer is a SuperSelective primer.

3. The method of claim 1, wherein the reverse primer comprises a 3′ region that is identical to the 5′ end of the target nucleic acid sequence and a 5′ region that is different from the 5′ end of the target nucleic acid sequence.

4. The method of claim 1, wherein between 1 and 10 linear amplification cycles are performed in step (b).

5. The method of claim 4, wherein 10 linear amplification cycles are performed in step (b).

6. The method of claim 1, wherein 1 low annealing temperature amplification cycle is performed in step (c).

7. The method of claim 1, wherein at least 30 LATE-PCR amplification cycles are performed in step (d).

8. The method of claim 1, wherein the melting temperature for the reverse primer on the target nucleic acid sequence is at least 5° C. lower than the melting temperature for the forward primer on the target nucleic acid sequence.

9. The method of claim 1, wherein the melting temperature for the reverse primer on the target nucleic acid sequence is at least 10° C. lower than the melting temperature for the forward primer on the target nucleic acid sequence.

10. The method of claim 1, wherein the reverse primer is present in the reaction solution at a concentration that is at least 2-fold higher than the concentration of the forward primer.

11. The method of claim 1, wherein the reverse primer is present in the reaction solution at a concentration that is at least 5-fold higher than the concentration of the forward primer.

12. The method of claim 1, wherein the reaction solution further comprises a reagent for detecting the formation of an amplification product in step (d).

13. The method of claim 12, wherein the detection reagent comprises a detectably labeled probe.

14. The method of claim 13, wherein the detection reagent is a molecular beacon probe.

15. The method of claim 12, wherein the detection reagent comprises a Lights-On probe and a Lights-Off probe or a Lights-Off Only probe and a dsDNA fluorescent dye.

16. The method of claim 12, further comprising the step of detecting the amplification product formed in step (d).

17. The method of claim 1, wherein the reaction solution comprises a Temperature Dependent Reagent.

18. The method of claim 17, wherein the method is performed using Temperature Imprecise PCR (TI-PCR).

19. A method of amplifying a target nucleic acid sequence in a target nucleic acid molecule comprising the steps of:
(a) forming a reaction solution comprising the target nucleic acid molecule, a forward primer, a reverse primer and amplification reagents;
(b) subjecting the reaction solution to conditions such that a linear amplification reaction is performed on the target nucleic acid molecule producing a first single-stranded amplification product comprising the forward primer and a sequence complementary to the target nucleic acid sequence;
(c) subjecting the reaction solution to conditions such that an exponential amplification reaction is performed on the first single-stranded amplification product producing a double-stranded nucleic acid amplification product comprising a first strand comprising the forward primer, a sequence complementary to the target nucleic acid sequence and a sequence complementary to the reverse primer, and comprising a second strand comprising the reverse primer, the target nucleic acid sequence and a sequence complementary to the forward primer; and
(d) subjecting the reaction solution to conditions such that a linear amplification reaction is performed on the first strand of the double-stranded amplification product producing a second single-stranded amplification product comprising the reverse primer, the target nucleic acid sequence and a sequence complementary to the forward primer.

20. The method of claim 19, wherein the forward primer has partial complementarity to nucleic acid sequence on the 3′ end of the target nucleic acid sequence and the reverse primer has partial identity to a nucleic acid sequence on the 5′ end of the target nucleic acid sequence.

21. The method of claim 20, wherein the melting temperature for the reverse primer on the target nucleic acid sequence is lower than the melting temperature for the forward primer on the target nucleic acid sequence.

22. The method of claim 19, wherein the reverse primer is present in the reaction solution at a higher concentration than the forward primer.

23. The method of claim 21, wherein step (b) comprises subjecting the reaction solution to one or more linear amplification cycles comprising an annealing temperature that is lower than the melting temperature of the forward primer on the target nucleic acid sequence and higher than the melting temperature of the reverse primer on the target nucleic acid sequence.

24. The method of claim 23, wherein steps (c) and (d) comprise subjecting the reaction solution to one or more low annealing temperature amplification cycles comprising an annealing temperature that is lower than the melting temperature of the reverse primer on the target nucleic acid sequence followed by subjecting the reaction solution to one or more LATE-PCR amplification cycles comprising an annealing temperature that is above the melting temperatures for the forward primer and the reverse primer on the target nucleic acid sequence and below the melting temperature for the forward primer and the reverse primer on perfectly complementary nucleic acid sequences.

25. The method of claim 20, wherein the forward primer is a SuperSelective primer.

26. The method of claim 20, wherein the reverse primer comprises a 3′ region that is identical to the 5′ end of the target nucleic acid sequence and a 5′ region that is different from the 5′ end of the target nucleic acid sequence.

27. The method of claim 23, wherein between 1 and 10 linear amplification cycles are performed in step (b).

28. The method of claim 23, wherein 10 linear amplification cycles are performed in step (b).

29. The method of claim 24, wherein 1 low annealing temperature amplification cycle is performed.

30. The method of claim 24, wherein at least 30 LATE-PCR amplification cycles are performed.

31. The method of claim 21, wherein the melting temperature for the reverse primer on the target nucleic acid sequence is at least 5° C. lower than the melting temperature for the forward primer on the target nucleic acid sequence.

32. The method of claim 21, wherein the melting temperature for the reverse primer on the target nucleic acid sequence is at least 10° C. lower than the melting temperature for the forward primer on the target nucleic acid sequence.

33. The method of claim 22, wherein the reverse primer is present in the reaction solution at a concentration that is at least 2-fold higher than the concentration of the forward primer.

34. The method of claim 22, wherein the reverse primer is present in the reaction solution at a concentration that is at least 5-fold higher than the concentration of the forward primer.

35. The method of claim 19, wherein the reaction solution further comprises a reagent for detecting the formation of the second single-stranded amplification product in step (d).

36. The method of claim 35, wherein the detection reagent comprises a detectably labeled probe.

37. The method of claim 36, wherein the detection reagent is a molecular beacon probe.

38. The method of claim 35, wherein the detection reagent comprises a Lights-On probe and a Lights-Off probe or a Lights-Off Only probe and a dsDNA fluorescent dye.

39. The method of claim 35, further comprising the step of detecting the second single-stranded amplification product formed in step (d).

40. The method of claim 19, wherein the reaction solution comprises a Temperature Dependent Reagent.

41. The method of claim 40, wherein the method is performed using Temperature Imprecise PCR (TI-PCR).

42. A kit for performing a Linear-Expo-Linear (LEL-PCR) amplification on a target nucleic acid sequence, the kit comprising a forward primer, a reverse primer and instructions for performing a LEL-PCR amplification, wherein
(i) the forward primer has partial complementarity to nucleic acid sequence on the 3′ end of the target nucleic acid sequence;
(ii) the reverse primer has partial identity to a nucleic acid sequence on the 5′ end of the target nucleic acid sequence; and
(iii) the melting temperature for the reverse primer on the target nucleic acid sequence is lower than the melting temperature for the forward primer on the target nucleic acid sequence.

43. The kit of claim 42, further comprising amplification reagents.

44. The kit of claim 42, further comprising a reagent for detecting a single-stranded amplification product.

45. The kit of claim 42, further comprising a Temperature Dependent Reagent.


SEQUENCE LISTING (43 sequences)

SEQ ID NO: 1 - DNA, 45 bp, Artificial Sequence
  Description of Artificial Sequence Synthetic primer
ctggtgaaaacaccgcagcatgtcgcacgagtgagccctgggcgg

SEQ ID NO: 2 - DNA, 24 bp, Artificial Sequence
  Description of Artificial Sequence Synthetic primer
gcatggtattctttctcttccgca

SEQ ID NO: 3 - DNA, 45 bp, Artificial Sequence
  Description of Artificial Sequence Synthetic primer
agacaactgttcaaactgatgggaaaacacaatcatctatttctc

SEQ ID NO: 4 - DNA, 24 bp, Artificial Sequence
  Description of Artificial Sequence Synthetic primer
agacaactgttcaaactgatggga

SEQ ID NO: 5 - DNA, 24 bp, Artificial Sequence
  Description of Artificial Sequence Synthetic primer
ctggtgaaaacaccgcagcatgtc

SEQ ID NO: 6 - DNA, 24 bp, Artificial Sequence
  Description of Artificial Sequence Synthetic primer
agacaactgttcaaactgatggga

SEQ ID NO: 7 - DNA, 22 bp, Artificial Sequence
  Description of Artificial Sequence Synthetic oligonucleotide
ggagcaaaatagcaatgaggta

SEQ ID NO: 8 - DNA, 22 bp, Artificial Sequence
  Description of Artificial Sequence Synthetic oligonucleotide
cctcgttttatcgttactccat

SEQ ID NO: 9 - DNA, 45 bp, Artificial Sequence
  Description of Artificial Sequence Synthetic primer
ctggtgaaaacaccgcagcatgtcgcccgagtgagccctgggcag

SEQ ID NO: 10 - DNA, 45 bp, Artificial Sequence
  Description of Artificial Sequence Synthetic primer
gaggaatgaaacggaggaagacgtacgtattctttctcttccgca

SEQ ID NO: 11 - DNA, 125 bp, Artificial Sequence
  Description of Artificial Sequence Synthetic polynucleotide
ctaaagccacctccttactttgcctccttctgcatggtattctttctcttccgcatgcaccagtttggcctgcccaaaatctgtgatcttgacatgctgcggtgttttcaccagtacgttcctgg

SEQ ID NO: 12 - DNA, 125 bp, Artificial Sequence
  Description of Artificial Sequence Synthetic polynucleotide
ctaaagccacctccttactttgcctccttctgcatggtattctttctcttccgcatgcaccagttcggcccgcccaaaatctgtgatcttgacatgctgcggtgttttcaccagtacgttcctgg

SEQ ID NO: 13 - DNA, 30 bp, Artificial Sequence
  Description of Artificial Sequence Synthetic oligonucleotide
cagctgcactgggaagggtgcagtctgacc

SEQ ID NO: 14 - DNA, 30 bp, Artificial Sequence
  Description of Artificial Sequence Synthetic oligonucleotide
ggtcagactgcacccttcccagtgcagctg

SEQ ID NO: 15 - DNA, 125 bp, Artificial Sequence
  Description of Artificial Sequence Synthetic polynucleotide
ctaaagccacctccttactttgcctccttctgcacggtattctttctcttccgcatgcaccagtttggcctgcccaaaatctgtgatcttgacatgctgcggtgttttcaccagtacgttcctgg

SEQ ID NO: 16 - DNA, 15 bp, Artificial Sequence
  Description of Artificial Sequence Synthetic probe
ccaaactggtgcggg

SEQ ID NO: 17 - DNA, 11 bp, Artificial Sequence
  Description of Artificial Sequence Synthetic oligonucleotide
gaataatatag

SEQ ID NO: 18 - DNA, 50 bp, Artificial Sequence
  Description of Artificial Sequence Synthetic oligonucleotide
gaataatatagccccccccccccccccccccccccccccctatattattc

SEQ ID NO: 19 - DNA, 49 bp, Artificial Sequence
  Description of Artificial Sequence Synthetic primer
tggccatggcaatcagttgctgttacctgtcaaaaggatactacacctc

SEQ ID NO: 20 - DNA, 48 bp, Artificial Sequence
  Description of Artificial Sequence Synthetic primer
aatctcctcctcctccttacctataaaaattttcggccaaggggatat

SEQ ID NO: 21 - DNA, 18 bp, Artificial Sequence
  Description of Artificial Sequence Synthetic probe
atccatatgataaattat

SEQ ID NO: 22 - DNA, 43 bp, Artificial Sequence
  Description of Artificial Sequence Synthetic primer
tggccagtcacagctataacatgtcaacgggaacagccaccaa

SEQ ID NO: 23 - DNA, 45 bp, Artificial Sequence
  Description of Artificial Sequence Synthetic primer
aatcctcctcctccttaaaaacttacggcccagtggaaattgatc

SEQ ID NO: 24 - DNA, 13 bp, Artificial Sequence
  Description of Artificial Sequence Synthetic probe
caggacagttttt

SEQ ID NO: 25 - DNA, 21 bp, Artificial Sequence
  Description of Artificial Sequence Synthetic oligonucleotide
gagcagactcgcactgaggta

SEQ ID NO: 26 - DNA, 21 bp, Artificial Sequence
  Description of Artificial Sequence Synthetic oligonucleotide
tacctcagtgcgagtctgctc

SEQ ID NO: 27 - DNA, 27 bp, Artificial Sequence
  Description of Artificial Sequence Synthetic oligonucleotide
gaggtgtagtatccttttgacaggtaa

SEQ ID NO: 28 - DNA, 41 bp, Artificial Sequence
  Description of Artificial Sequence Synthetic primer
tcgtgaatacctccagctcggcaccctcacgtgacagaccg

SEQ ID NO: 29 - DNA, 35 bp, Artificial Sequence
  Description of Artificial Sequence Synthetic primer
cgagtccatcacctcgcgatcacaccacagacgtt

SEQ ID NO: 30 - DNA, 23 bp, Artificial Sequence
  Description of Artificial Sequence Synthetic probe
tcaggtccatgaattggctcaga

SEQ ID NO: 31 - DNA, 11 bp, Artificial Sequence
  Description of Artificial Sequence Synthetic oligonucleotide
ctatattattc

SEQ ID NO: 32 - DNA, 3519 bp, Mycobacterium tuberculosis
ttggcagattcccgccagagcaaaacagccgctagtcctagtccgagtcgcccgcaaagttcctcgaataactccgtacccggagcgccaaaccgggtctccttcgctaagctgcgcgaaccacttgaggttccgggactccttgacgtccagaccgattcgttcgagtggctgatcggttcgccgcgctggcgcgaatccgccgccgagcggggtgatgtcaacccagtgggtggcctggaagaggtgctctacgagctgtctccgatcgaggacttctccgggtcgatgtcgttgtcgttctctgaccctcgtttcgacgatgtcaaggcacccgtcgacgagtgcaaagacaaggacatgacgtacgcggctccactgttcgtcaccgccgagttcatcaacaacaacaccggtgagatcaagagtcagacggtgttcatgggtgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatgcgatcgacgctggagaaggacaacaccgtcggcaccgacgaggcgctgttggacatctaccgcaagctgcgtccgggcgagcccccgaccaaagagtcagcgcagacgctgttggaaaacttgttcttcaaggagaagcgctacgacctggcccgcgtcggtcgctataaggtcaacaagaagctcgggctgcatgtcggcgagcccatcacgtcgtcgacgctgaccgaagaagacgtcgtggccaccatcgaatatctggtccgcttgcacgagggtcagaccacgatgaccgttccgggcggcgtcgaggtgccggtggaaaccgacgacatcgaccacttcggcaaccgccgcctgcgtacggtcggcgagctgatccaaaaccagatccgggtcggcatgtcgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgcacccgtcgcactacggccggatgtgcccgatcgaaacccctgaggggcccaacatcggtctgatcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccaccgcgatgattcccttcctggagcacgacgacgccaaccgtgccctcatgggggcaaacatgcagcgccaggcggtgccgctggtccgtagcgaggccccgctggtgggcaccgggatggagctgcgcgcggcgatcgacgccggcgacgtcgtcgtcgccgaagaaagcggcgtcatcgaggaggtgtcggccgactacatcactgtgatgcacgacaacggcacccggcgtacctaccggatgcgcaagtttgcccggtccaaccacggcacttgcgccaaccagtgccccatcgtggacgcgggcgaccgagtcgaggccggtcaggtgatcgccgacggtccctgtactgacgacggcgagatggcgctgggcaagaacctgctggtggccatcatgccgtgggagggccacaactacgaggacgcgatcatcctgtccaaccgcctggtcgaagaggacgtgctcacctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacatcggccagattttggagacccacctgggttggtgtgcccacagcggctggaaggtcgacgccgccaagggggttccggactgggccgccaggctgcccgacgaactgctcgaggcgcagccgaacgccattgtgtcgacgccggtgttcgacggcgcccaggaggccgagctgcagggcctgttgtcgtgcacgctgcccaaccgcgacggtgacgtgctggtcgacgccgacggcaaggccatgctcttcgacgggcgcagcggcgagccgttcccgtacccggtcacggttggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgctgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggtgctatcgagtgacggtgcggcgatcgaactgcgcgaaggtgaggacgaggacctggagcgggccgcggccaacctgggaatcaatctgtcccgcaacgaatccgcaagtgtcgaggatcttgcgtaa

SEQ ID NO: 33 - DNA, 3519 bp, Mycobacterium bovis
ttggcagattcccgccagagcaaaacagccgctagtcctagtccgagtcgcccgcaaagttcctcgaataactccgtacccggagcgccaaaccgggtctccttcgctaagctgcgcgaaccacttgaggttccgggactccttgacgtccagaccgattcgttcgagtggctgatcggttcgccgcgctggcgcgaatccgccgccgagcggggtgatgtcaacccagtgggtggcctggaagaggtgctctacgagctgtctccgatcgaggacttctccgggtcgatgtcgttgtcgttctctgaccctcgtttcgacgatgtcaaggcacccgtcgacgagtgcaaagacaaggacatgacgtacgcggctccactgttcgtcaccgccgagttcatcaacaacaacaccggtgagatcaagagtcagacggtgttcatgggtgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatgcgatcgacgctggagaaggacaacaccgtcggcaccgacgaggcgctgttggacatctaccgcaagctgcgtccgggcgagcccccgaccaaagagtcagcgcagacgctgttggaaaacttgttcttcaaggagaagcgctacgacctggcccgcgtcggtcgctataaggtcaacaagaagctcgggctgcatgtcggcgagcccatcacgtcgtcgacgctgaccgaagaagacgtcgtggccaccatcgaatatctggtccgcttgcacgagggtcagaccacgatgaccgttccgggcggcgtcgaggtgccggtggaaaccgacgacatcgaccacttcggcaaccgccgcctgcgtacggtcggcgagctgatccaaaaccagatccgggtcggcatgtcgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgcacccgtcgcactacggccggatgtgcccgatcgaaacccctgaggggcccaacatcggtctgatcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccaccgcgatgattcccttcctggagcacgacgacgccaaccgtgccctcatgggggcaaacatgcagcgccaggcggtgccgctggtccgtagcgaggccccgctggtgggcaccgggatggagctgcgcgcggcgatcgacgccggcgacgtcgtcgtcgccgaagaaagcggcgtcatcgaggaggtgtcggccgactacatcactgtgatgcacgacaacggcacccggcgtacctaccggatgcgcaagtttgcccggtccaaccacggcacttgcgccaaccagtgccccatcgtggacgcgggcgaccgagtcgaggccggtcaggtgatcgccgacggtccctgtactgacgacggcgagatggcgctgggcaagaacctgctggtggccatcatgccgtgggagggccacaactacgaggacgcgatcatcctgtccaaccgcctggtcgaagaggacgtgctcacctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacatcggccagattttggagacccacctgggttggtgtgcccacagcggctggaaggtcgacgccgccaagggggttccggactgggccgccaggctgcccgacgaactgctcgaggcgcagccgaacgccattgtgtcgacgccggtgttcgacggcgcccaggaggccgagctgcagggcctgttgtcgtgcacgctgcccaaccgcgacggtgacgtgctggtcgacgccgacggcaaggccatgctcttcgacgggcgcagcggcgagccgttcccgtacccggtcacggttggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgccgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggtgctatcgagtgacggtgcggcgatcgaactgcgcgaaggtgaggacgaggacctggagcgggccgcggccaacctgggaatcaatctgtcccgcaacgaatccgcaagtgtcgaggatcttgcgtaa

SEQ ID NO: 34 - DNA, 3519 bp, Mycobacterium africanum
ttggcagattcccgccagagcaaaacagccgctagtcctagtccgagtcgcccgcaaagttcctcgaataactccgtacccggagcgccaaaccgggtctccttcgctaagctgcgcgaaccacttgaggttccgggactccttgacgtccagaccgattcgttcgagtggctgatcggttcgccgcgctggcgcgaatccgccgccgagcggggtgatgtcaacccagtgggtggcctggaagaggtgctctacgagctgtctccgatcgaggacttctccgggtcgatgtcgttgtcgttctctgaccctcgtttcgacgatgtcaaggcacccgtcgacgagtgcaaagacaaggacatgacgtacgcggctccactgttcgtcaccgccgagttcatcaacaacaacaccggtgagatcaagagtcagacggtgttcatgggtgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatgcgatcgacgctggagaaggacaacaccgtcggcaccgacgaggcgctgttggacatctaccgcaagctgcgtccgggcgagcccccgaccaaagagtcagcgcagacgctgttggaaaacttgttcttcaaggagaagcgctacgacctggcccgcgtcggtcgctataaggtcaacaagaagctcgggctgcatgtcggcgagcccatcacgtcgtcgacgctgaccgaagaagacgtcgtggccaccatcgaatatctggtccgcttgcacgagggtcagaccacgatgatcgttccgggcggcgtcgaggtgccggtggaaaccgacgacatcgaccacttcggcaaccgccgcctgcgtacggtcggcgagctgatccaaaaccagatccgggtcggcatgttgcggatggagcgggtggtccgggagcggatgaccacccaggacgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggaggtccgcgacgtgcacccgtcgcactacggccggatgtgcccgatcgaaacccctgaggggcccaacatcggtctgatcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccaccgcgatgattcccttcctggagcacgacgacgccaaccgtgccctcatgggggcaaacatgcagcgccaggcggtgccgctggtccgtagcgaggccccgctggtgggcaccgggatggagctgcgcgcggcgatcgacgccggcgacgtcgtcgtcgccgacgaaagcggcgtcatcgaggaggtgtcggccgactacatcactgtgatgcacgacaacggcacccggcgtacctaccggatgcgcaagtttgcccggtccaaccacggcacttgcgccaaccagtgccccatcgtggacgcgggcgaccgagtcgaggccggtcaggtgatcgccgacggtccctgtactgacgacggcgagatggcgctgggcaagaacctgctggtggccatcatgccgtgggagggccacaactacgaggacgcgatcatcctgtccaaccgcctggtcgaagaggacgtgctcacctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacatcggccagattttggagacccacctgggttggtgtgcccacagcggctggaaggtcgacgccgccaagggggttccggactgggccgccaggctgcccgacgaactgctcgaggcgcagccgaacgccattgtgtcgacgccggtgttcgacggcgcccaggaggccgagctgcagggcctgttgtcgtgcacgctgcccaaccgcgacggtgacgtgctggtcgacgccgacggcaaggccatgctcttcgacgggcgcagcggcgagccgttcccgtacccggtcacggttggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgccgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggtgctatcgagtgacggtgcggcgatcgaactgcgcgaaggtgaggacgaggacctggagcgggccgcggccaacctgggaatcaatctgtcccgcaacgaatccgcaagtgtcgaggatcttgcgtaa

SEQ ID NO: 35 - DNA, 3519 bp, Mycobacterium canettii
ttggcagattcccgccagagcaaaacagccgctagtcctagtccgagtcgcccgcaaagtttctcgaataactccgtacccggagcgccaaaccgggtctccttcgctaagctgcgcgaaccacttgaggttccgggactccttgacgtccagaccgattcgttcgagtggctgatcggttcgccgcgctggcgcgaatccgccgccgagcggggtgatgtcaacccggtgggtggcctggaagaggtgctctacgagctgtctccgatcgaggacttctccgggtcgatgtcgttgtcgttctctgaccctcgtttcgacgatgtcaaggcacccgtcgacgagtgcaaagacaaggacatgacgtacgcggctccactgttcgtcaccgccgagttcatcaacaacaacaccggtgagatcaagagtcagacggtgttcatgggcgacttcccgatgatgaccgagaagggcacgttcatcatcaacgggaccgagcgtgtggtggtcagccagctggtgcggtcgcccggggtgtacttcgacgagaccattgacaagtccaccgacaagacgctgcacagcgtcaaggtgatcccgagccgcggcgcgtggctcgagtttgacgtcgacaagcgcgacaccgtcggcgtgcgcatcgaccgcaaacgccggcaaccggtcaccgtgctgctcaaggcgctgggctggaccagcgagcagattgtcgagcggttcgggttctccgagatcatgcgatcgacgctggagaaggacaacaccgtcggcaccgacgaggcgctgttggacatctaccgcaagctgcgtccgggcgagcccccgaccaaagagtcagcgcagacgctgttggaaaacttgttcttcaaggagaagcgctacgaccttgcccgggtaggtcgctacaaggtcaacaagaagctcggcctgcatgtcggcgagcccatcacgtcgtcgacgctgaccgaagaagacgtcgtggccaccatcgaatatctggtccgcttgcacgagggtcagaccacgatgaccgttccgggcggcgtcgaggtgccggtggaaaccgacgacatcgaccacttcggcaaccgccgcctgcgtacggtcggcgagctgatccaaaaccagatccgggtcggcatgtcgcggatggagcgggtggttcgggagcggatgaccacccaggacgtggaggcgatcacaccgcagacgttgatcaacatccgcccggtggtcgccgcgatcaaggagtttttcggcaccagccagctgagccaattcatggaccagaacaacccgctgtcggggttgacccacaagcgccgactgtcggcgctggggcccggcggtctgtcacgtgagcgtgccgggctggaggtccgcgatgtgcacccgtcgcactacggccggatgtgcccgatcgagacccctgaggggcccaacatcggtctgatcggatcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaactcgccgatcgatgccgacggtcgcttcgtcgagccgcgcgtgctggtccgccgcaaggcgggcgaggtggagtacgtgccctcgtccgaggtggactacatggacgtctcgccccgccagatggtgtcggtggccaccgcgatgattcccttcctggagcacgacgacgccaaccgtgcactcatgggggccaacatgcagcgccaggcggtgccgctggtccgtagcgaggccccgctggtgggcaccgggatggagctgcgcgcggcgatcgacgccggcgacgtcgtcgtcgccgaagaaagcggcgtcatcgaggaggtgtcggccgactacatcactgtgatgcacgacaacggcacccggcgtacctaccggatgcgcaagtttgcccggtccaaccacggcacctgcgccaaccagtgccccatcgtggacgcgggcgaccgagtcgaggccggtcaggtgatcgccgacggtccctgtactgacgacggcgagatggcgctgggcaagaacctgctggtggccatcatgccgtgggagggccacaactacgaggacgcgatcatcctgtccaaccgcctggtcgaagaggacgtgctcacctcgatccacatcgaggagcatgagatcgatgctcgcgacaccaagctgggtgcggaggagatcacccgcgacatcccgaacatctccgacgaggtgctcgccgacctggatgagcggggcatcgtgcgcatcggtgccgaggttcgcgacggggacatcctggtcggcaaggtcaccccgaagggtgagaccgagctgacgccggaggagcggctgctgcgtgccatcttcggtgagaaggcccgcgaggtgcgcgacacttcgctgaaggtgccgcacggcgaatccggcaaggtgatcggcattcgggtgttttcccgcgaggacgaggacgagttgccggccggtgtcaacgagctggtgcgtgtgtatgtggctcagaaacgcaagatctccgacggtgacaagctggccggccggcacggcaacaagggcgtgatcggcaagatcctgccggttgaggacatgccgttccttgccgacggcaccccggtggacattattttgaacacccacggcgtgccgcgacggatgaacatcggccagattttggagacccacctgggttggtgtgcccacagcggctggaaggtcgacgccgccaagggggttccggactgggccgccaggctgcccgacgaactgctcgaggcgcagccgaacgccattgtgtcgacgccggtgttcgacggcgcccaggaggccgagctgcagggcctgttgtcggccacgctgcccaaccgcgacggtgacgtgctggtcgacgccgacggcaaggccatgctcttcgacgggcgcagcggcgagccgttcccgtacccggtcacggttggctacatgtacatcatgaagctgcaccacctggtggacgacaagatccacgcccgctccaccgggccgtactcgatgatcacccagcagccgctgggcggtaaggcgcagttcggtggccagcggttcggggagatggagtgctgggccatgcaggcctacggtgccgcctacaccctgcaggagctgttgaccatcaagtccgatgacaccgtcggccgcgtcaaggtgtacgaggcgatcgtcaagggtgagaacatcccggagccgggcatccccgagtcgttcaaggtgctgctcaaagaactgcagtcgctgtgcctcaacgtcgaggtgctatcgagtgacggtgcggcgatcgaactgcgcgaaggtgaggacgaggacctggagcgggccgcggccaacctgggaatcaatctgtcccgcaacgaatccgcaagtgtcgaggatcttgcgtaa

SEQ ID NO: 36 - DNA, 191 bp, Mycobacterium tuberculosis
acgtggaggcgatcacaccgcagacgttgatcaacatccggccggtggtcgccgcgatcaaggagttcttcggcaccagccagctgagccaattcatggaccagaacaacccgctgtcggggttgacccacaagcgccgactgtcggcgctggggcccggcggtctgtcacgtgagcgtgccgggctggag

SEQ ID NO: 37 - DNA, 201 bp, Mycobacterium abscessus
accacgcaggacgtcgaggcgatcaccccgcagaccctgatcaacatccgtcccgtcgtggcggcgatcaaggagttcttcggaaccagccagctgtcgcagttcatggaccagaacaacccgctgtcgggcctgacccacaagcgtcgtctgtcggcgctgggccccggtggtctgacccgtgaccgcgccggcctcgag

SEQ ID NO: 38 - DNA, 191 bp, Mycobacterium canettii
acgtggaggcgatcacaccgcagacgttgatcaacatccgcccggtggtcgccgcgatcaaggagtttttcggcaccagccagctgagccaattcatggaccagaacaacccgctgtcggggttgacccacaagcgccgactgtcggcgctggggcccggcggtctgtcacgtgagcgtgccgggctggag

SEQ ID NO: 39 - DNA, 173 bp, Mycobacterium chelonae
cgcagaccctgatcaacatccgtcccgtcgtggcggcgatcaaggagttcttcggaaccagccagctgtcgcagttcatggaccagaacaacccgctgtcgggtctgacccacaagcgtcgtctgtcggcgctgggccccggtggtctgactcgtgaccgcgccggccttgag

SEQ ID NO: 40 - DNA, 200 bp, Mycobacterium flavescens
accacccaggacgtcgaggcgatcacgccgcagaccctgatcaacatccgtcccgtcgtggcggcgatcaaggagttcttcggtacgtcgcagctgtcgcagttcatggaccagaacaacccgctgtcgggtctgacccacaagcgccgcctgtcggcgctgggccccggtggtctgtcccgtgagcgcgccggcctcga

SEQ ID NO: 41 - DNA, 197 bp, Mycobacterium fortuitum
cccaggacgtggaggcgatcacaccgcagaccctgatcaacatccgtcccgtcgtggcggcgatcaaggagttcttcggaacgtcgcagctgtcgcagttcatggatcagaacaacccgctgtcgggtctgacccacaagcgtcgtctgtcggcgctgggccccggcggtctgtcccgtgagcgcgccggccttgag

SEQ ID NO: 42 - DNA, 196 bp, Mycobacterium gordonae
cccaggacgtggaggcgatcacaccgcagaccctgatcaacatccggccggtcgtcgccgcgatcaaggagttcttcggcaccagccagctctcgcagttcatggaccagaacaacccgctgtcgggtctgacccacaagcgtcgtctgtcggcgctggggccgggtggtctgtcccgtgagcgtgcgggtctgga

SEQ ID NO: 43 - DNA, 201 bp, Mycobacterium xenopi
accactcaggacgtcgaggcgatcaccccgcagaccttgatcaacatccgccccgtggtggccgcgatcaaggagttcttcggcaccagccagctctcgcagttcatggatcagaacaacccgctgtcggggctcacccacaagcggcggctctcggcgcttggtccgggcggtctgtcgcgcgagcgggccgggctggag